SEQUENCE LISTING (72 sequences)

SEQ ID NO: 1 - DNA, 23 bp, artificial
  a primer for HPV 16 F
gcacaggaagcaaaacaacatag

SEQ ID NO: 2 - DNA, 21 bp, artificial
  a primer for HPV 18 F
aggtccacaatgatgcacaag

SEQ ID NO: 3 - DNA, 24 bp, artificial
  a primer for HPV 33/33n F
cagaagatgaggatgaaacagcag

SEQ ID NO: 4 - DNA, 20 bp, artificial
  a primer for HPV 31 F
gaggaacatgcagaggctgt

SEQ ID NO: 5 - DNA, 20 bp, artificial
  a primer for HPV 35 F
atgcacaggaggagcaaaca

SEQ ID NO: 6 - DNA, 24 bp, artificial
  a primer for HPV 39 F
gtgagacagcacaggtacttttac

SEQ ID NO: 7 - DNA, 19 bp, artificial
  a primer for HPV 45 F
ccatgcgcaggaagttcag

SEQ ID NO: 8 - DNA, 20 bp, artificial
  a primer for HPV 51 F
acaaagaggctgtgcatcag

SEQ ID NO: 9 - DNA, 23 bp, artificial
  a primer for HPV 52 F
gaaggggaggatgatttacatgc

SEQ ID NO: 10 - DNA, 21 bp, artificial
  a primer for HPV 56/66 F
gcaagtacaaacagcacatgc

SEQ ID NO: 11 - DNA, 20 bp, artificial
  a primer for HPV 58 F
agcccgagcgttgtttaatg

SEQ ID NO: 12 - DNA, 23 bp, artificial
  a primer for HPV 59 F
ggccttgtttaatgtgcaggaag

SEQ ID NO: 13 - DNA, 22 bp, artificial
  a primer for HPV 68 F
caaacaggtgacacagtctcag

SEQ ID NO: 14 - DNA, 21 bp, artificial
  a primer for HPV 16/31/33/33n/35/39/52/56/58/66 R
gtattgccatacccgctgtct

SEQ ID NO: 15 - DNA, 22 bp, artificial
  a primer for HPV 18/45 R
aacagccatagccactatctga

SEQ ID NO: 16 - DNA, 21 bp, artificial
  a primer for HPV 51 R
ctgtccggataactgtccagt

SEQ ID NO: 17 - DNA, 24 bp, artificial
  a primer for HPV 59 R
tgtctggcactgttaktaaccttc

SEQ ID NO: 18 - DNA, 21 bp, artificial
  a primer for HPV 68 R
tcctgtaatggcgactttgct

SEQ ID NO: 19 - DNA, 25 bp, artificial
  a primer for HPV 16 F HEX
taatggatggttttatgtagaggct

SEQ ID NO: 20 - DNA, 24 bp, artificial
  a primer for HPV 16 R HEX
ctgcctgtgttaaataatcattat

SEQ ID NO: 21 - DNA, 21 bp, artificial
  a primer for HPV 18 F HEX
ggctggttttatgtacaagct

SEQ ID NO: 22 - DNA, 21 bp, artificial
  a primer for HPV 18 R HEX
cctgttcacaaaatgttcctt

SEQ ID NO: 23 - DNA, 24 bp, artificial
  a primer for HBE1 IC F
gagaacttcaaggtgagttcaggt

SEQ ID NO: 24 - DNA, 24 bp, artificial
  a primer for HBE1 IC R
gaagtctgctgttctaacatcaag

SEQ ID NO: 25 - DNA, 10 bp, artificial
  a sequence of HR-HPV
actttcgttt

SEQ ID NO: 26 - DNA, 10 bp, artificial
  a sequence of HPV 33n
actttcgctt

SEQ ID NO: 27 - DNA, 15 bp, artificial
  a sequence of HPV 16/18
atatcwgatgacgag

SEQ ID NO: 28 - DNA, 30 bp, artificial
  a sequence of HBE1
tgacattaattgaagctcataatcttattg

SEQ ID NO: 29 - DNA, 10 bp, artificial
  a probe for HR-HPV
  misc_feature (1)..(1): 5′-6FAM
  misc_feature (10)..(10): BHQ-1-3′
actttcgttt

SEQ ID NO: 30 - skipped

SEQ ID NO: 31 - DNA, 15 bp, artificial
  a probe for 16/18
  misc_feature (1)..(1): 5′-HEX
  misc_feature (6)..(6): “W” signifies a change of base A/T.
  misc_feature (15)..(15): BHQ-1-3′
atatcwgatgacgag

SEQ ID NO: 32 - DNA, 30 bp, artificial; identical to SEQ ID NO: 28
  a probe for IC
  misc_feature (1)..(1): 5′-ROX
  misc_feature (30)..(30): BHQ-2-3′

SEQ ID NO: 33 - DNA, 180 bp, artificial
  region of HPV16 genome
gcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcgaagacagc

SEQ ID NO: 34 - DNA, 10 bp, artificial
  consensus region of HPV genome targeted by pan-HPV probe
aaacgaaagt

SEQ ID NO: 35 - DNA, 240 bp, artificial
  region of HPV 33n genome
cattgtttaatatacaggaaggggaggatgacttaaatgctgtgtgtgcactaaagcgaaagtttgccgcatgttcacaaagtgctgcggaggacgttgttgatcgtgctgcaaacccgtgtagaacgtctattaataaaaataaagaatgcacatacagaaaacgaaaaatagatgagctagaagacagcggatatggcaatactgaagtggaaactcagcagatggtacaacaggtag

SEQ ID NO: 36 - DNA, 10 bp, artificial
  region of HPV 33n genome targeted by 33n probe
aagcgaaagt

SEQ ID NO: 37 - DNA, 240 bp, artificial
  region of HPV 16 genome
acgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttgggt

SEQ ID NO: 38 - DNA, 15 bp, artificial
  HPV 16 probe
atatcagatgacgag

SEQ ID NO: 39 - DNA, 15 bp, artificial
  HPV 18 probe
atatctgatgacgag

SEQ ID NO: 40 - DNA, 420 bp, artificial
  Region of HPV 18 genome
gcagtaagcaacaatggctgatccagaaggtacagacggggagggcacggggtgtaacggctggttttatgtacaagctattgtagacaaaaaaacaggagatgtaatatctgatgacgaggacgaaaatgcaacagacacagggtcggatatggtagattttattgatacacaaggaacattttgtgaacaggcagagctagagacagcacaggcattgtttcatgcgcaggaggtccacaatgatgcacaagtgttgcatgttttaaaacgaaagtttgcaggaggcagcaaagaaaacagtccattaggggagcggctggaggtggatacagagttaagtccacggttacaacaaatatcattaaatagtgggcaaaaaaaggcaaaaaggcggctgttcacaatatcagatagtgg

SEQ ID NO: 41 - DNA, 240 bp, artificial
  Region of HPV 16 genome
gcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgag

SEQ ID NO: 42 - DNA, 10 bp, artificial
  Reverse complement pan-HPV probe sequence, which corresponds to the consensus region targeted by the pan-HPV probe
aaacgaaagt

SEQ ID NO: 43 - DNA, 194 bp, artificial
  Example PCR product obtained from primers HPV 16 F and common reverse primer
gcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcgaagacagcgggtatggcaatac

SEQ ID NO: 44 - DNA, 1950 bp, artificial
  HPV 16 nucleotides 865-2814 E1 gene sequence
atggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatga

SEQ ID NO: 45 - DNA, 21 bp, artificial
  region of the HPV 16 E1 gene targeted by the reverse primer HPV 16/31/33/33n/35/39/52/56/58/66R
agacagcgggtatggcaatac

SEQ ID NO: 46 - DNA, 1974 bp, artificial
  HPV 18 nucleotides 914 - 2887 E1 gene sequence
atggctgatccagaaggtacagacggggagggcacggggtgtaacggctggttttatgtacaagctattgtagacaaaaaaacaggagatgtaatatctgatgacgaggacgaaaatgcaacagacacagggtcggatatggtagattttattgatacacaaggaacattttgcgaacaggcagagctagagacagcacaggcattgtttcatgcgcaggaggtccacaatgatgcacaagtgttgcatgttttaaaacgaaagtttgcaggaggcagcaaggaaaacagtccattaggggagcggctggaggtggatacagagttaagtccacggttacaacaaatatcattaaatagtgggcaaaaaaaggcaaaaaggcggctgttcacaatatcagatagtggctatggctgttctgaagtggaagcaacacagattcaggtaattacaaatggcgaacagggcggcaatgtatgtagtggcggcagtacagaggctatagacaatgggggcacagagggcaacaacagcagtgtagacggtacaagtgacactagcaatatagaaaatgtaaatccacaatgtaccatagcacaattaaaagacttgttaaaagtaaacaataaacaaggagcaatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtcttgactgtaaatggggagtattaatattagccctgttgcgttacaaatgtggtaaaagtagactaacagttgctaaaggtttaagtacgttgttacacgtacctgaaacttgtatgttaattcaaccaccaaaattgcgaagtagtgttgcagcactgtattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcggctgcctttttaaaaagcaattgccaagctaaatatttaaaagattgtgccacaatgtgcaaacattataggcgagcccaaaaacgacaaatgaatatgtcacagtggatacgatttagatgtttaaaaatagatgaagggggagattggagaccaatagtgcaatttttgcgataccaacaaatagagtttataacatttttaggagccttaaaatcatttttaaaaggaactcccaaaaaaaattgtttagtattctgtggaccagcaaatacaggaaaatcatattttggaatgagttttatacactttatacaaggagcagtaatatcatttgtaaattctactagtcatttttggttggaaccgttaacagataccaaggtggccatgttagatgatgcaacaaccacgtgttggacatactttgatacctatatgagaaatgctttagatggcaatcccataagtattgatagaaagcacaaaccattaatacaactaaaatgtcctccaatactactaaccacaaatatacatccagcaaaggataacagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagtgcgttgcaggacaaaatcatagaccactatga

SEQ ID NO: 47 - DNA, 22 bp, artificial
  Region of HPV18 E1 genome targeted by reverse primer HPV 18/45 R
tcagatagtggctatggctgtt

SEQ ID NO: 48 - DNA, 2002 bp, artificial
  HPV 33 genome comprising nucleotides 879 - 2813 E1 gene sequence
atggccgatcctgaaggtacaaatggggctgggatggggtgtactggttggtttgaggtagaagcagtcatagagagaagaacaggagataatatttcagaagatgaggatgaaacagcagatgacagtggcacggatttactagagtttatagatgattctatggaaaatagtatacaggcagacacagaggcagcccgggcattgtttaatatacaggaaggggaggatgacttaaatgctgtgtgtgcactaaagcgaaagtttgccgcatgttcacaaagtgctgcggaggacgttgttgatcgtgctgcaaacccgtgtagaacgtctattaataaaaataaagaatgcacatacagaaaacgaaaaatagatgagctagaagacagcggatatggcaatactgaagtggaaactcagcagatggtacaacaggtagaaagtcaaaatggcgacacaaacttaaatgacttagaatctagtggggtgggggatgattcagaagtaagctgtgagacaaatgtagatagctgtgaaaatgttacattgcaggaaattagtaatgttctacatagtagtaatacaaaagcaaatatattatataaatttaaagaggcctatggaataagttttatggaattagtaagaccatttaaaagtgataaaacaagctgtacagattggtgtataacaggatatggaattagtccatcagtagcagaaagtttaaaagtattaattaaacagcatagtttgtatactcatttacaatgtttaacttgcgatagaggaataataatattattgttaattagatttaggtgtagcaaaaacaggttaacagtagcaaaactaatgagtaatttattatcaatacctgaaacatgtatggttatagagccaccaaagttacggagccaaacatgtgcattgtattggtttagaacagcaatgtcaaacattagtgatgtacaaggtacaacacctgaatggatagatagactaactgttttacaacatagctttaatgataatatatttgatttaagtgaaatggtacagtgggcatacgataacgagttaacggacgatagtgacattgcatattattatgcacaacttgcagattcaaatagtaatgctgctgcatttttaaaaagtaactcacaagcaaaaatagtaaaggactgtggaataatgtgtagacattataaaaaagcagaaaaacgtaaaatgtcaataggacaatggatacaaagtagatgtgaaaaaacaaatgatggaggaaattggagaccaatagtacagttgttaagatatcaaaacattgaatttacagcatttttaggtgcatttaaaaagtttttaaaaggtataccaaaaaaaagctgtatgctaatttgtggaccagcaaatacaggaaagtcatattttggaatgagtttaatacagtttttaaaagggtgtgttatatcatgtgtaaattctaaaagtcacttttggttgcagccattatcagatgcaaaaataggaatgatagatgatgtaacgccaataagttggacatatatagatgattacatgagaaatgcgttagatggaaatgaaatttcaatagatgtgaaacatagggcattagtgcaattaaaatgtccaccactgcttcttacctcaaatacaaatgcaggcacagactctagatggccatatttacatagtagattaacagtatttgaatttaaaaatccattcccatttgatgaaaatggtaacccagtgtatgtaataaatgatgaaaattggaaatcctttttctcaaggacgtggtgcaaattagatttaatagaggaagaggacaaggaaaaccatggaggaaatatcagcacgtttaaatgcagtgcaggagaaaatactagatctttacgaagctgataaaactgatttaccatcacaaattgaacattggaaactgatacgcatggagtgtgctttattttat

SEQ ID NO: 49 - DNA, 21 bp, artificial
  HPV 39 sequence targeted by the Reverse primer HPV16/31/33/33n/35/39/52/56/58/66R
agacagcggatatggcaatac

SEQ ID NO: 50 - DNA, 21 bp, artificial
  Reverse complement of SEQ ID NO: 49
gtattgccatatccgctgtct

SEQ ID NO: 51 - DNA, 1891 bp, artificial
  HPV31 genome sequence comprising nucleotides 862 - 2751 E1 gene sequence
aatggctgatccagcaggtacagatggggaggggacgggatgcaatggttggttttatgtagaagcagtaattgacagacagacaggggacaacatttcagacgacgaaaatgaagacagtagtgatactggggaggatatggttgactttattgacaattgtaatgtatacaacaatcaggcagaagcagagacagcacaggcattgtttcatgcacaggaagcggaggaacatgcagaggctgtgcaggttctaaaacgaaagtatgtaggtagtcctttaagtgatattagtagttgtgtggattataatattagtccacggttaaaagctatatgcatagaaaataacagtaaaacagcaaaacgaagactttttgaacttccagacagcgggtatggcaatactgaagtggaagcgcagcagatggtacaggtagaggagcaacaaacaacattaagttgtaatggtagtgacgggacacatagtgaacgagacaatgaaactccaacacgtaatatattgcaagtgttaaaaactagcaatggtaaagctgctatgttaggtaaatttaaagaattatatggtgtaagttttatggaactaattaggccatttcaaagcaataaaagcacatgtgctgattggtgtgtagctgcgtttggagttacaggtacagttgcagaaggatttaaaaccctattgcaaccatattgtttgtattgccatttacaaagtttagcatgttcctggggcatggttatgttaatgcttgtaagatttaaatgtgcaaaaaatagaataacaattgaaaaattattagaaaagttattgtgtatatctacaaattgtatgttaattcagccacccaaattacgtagtacagctgcagcattatattggtacagaacaggaatgtcaaacattagtgatgtatatggtgaaacaccagaatggatagaaagacaaacagtattacagcatagttttaatgacacaacatttgatttgtcccaaatggtacaatgggcatatgacaatgatgttatggatgatagtgaaattgcctatagatatgcacaattagctgacagtgatagtaatgcatgtgcatttttaaaaagtaattcgcaggcaaaaatagttaaagattgtggaacaatgtgtagacattataaacgagcagaaaaacgacaaatgtctatgggacagtggattaaaagtagatgtgacaaagttagtgacgaaggtgactggagggacatagtaaagtttttaagatatcaacaaatagaatttgtgtcatttttatctgcattaaagctgtttttaaaaggagtgccaaagaaaaactgtattttaatacatggtgcacctaatacaggtaaatcatattttggaatgagccttatgagcctcttacaaggatgtataatatcatatgcaaattcaaaaagtcatttttggttacaaccactggctgatgctaaaataggcatgttagatgatgctacaacgccatgttggcattatatagacaattacctacgaaatgcactagatggcaaccctgtatctatagatgtaaagcataaagctttaatgcagttaaaatgtcctcctttattgattacatctaatataaatgcaggtaaggatgacagatggccatacctacatagcagactagtggtttttacatttccaaatccatttccatttgacaaaaacggaaatccagtatatgaattaagtgataaaaactggaaatcctttttctcaaggacgtggtgcagattaaatttgcacgaggaagaggacaaagaaaacgatggagactctttctcaacgtttaaatgtgtgtcaggacaaaatattagaacattatga

SEQ ID NO: 52 - DNA, 1914 bp, artificial
  HPV35 nucleotides 868 - 2781 E1 gene sequence
atggctgatcctgcaggtacagatgaaggggaggggacgggatgtaatggatggttttttgtagaagcagtagttagtagacgtacgggggatccagtgtcagaggacgaaaatgaagatgactgtgacaggggggaggatatggtggactttataaatgatacagatatattaaacatacaggcagaaacagagacagcacaagcattatttcatgcacaggaggagcaaacacacaaagaggctgtacaggtcctaaaacgaaagtatgctagtagtccacttagcagcgtgagcttatgtgttaataataacataagtccacgtttaaaagctatttgcattgaaaataaaaatacagcagcaaagcgacgattatttgaactaccagacagcggttatggcaattctgaagtggaaatacagcagatacaacaggtagaggggcatgatacagttgaacaatgtagtatgggcagtggggatagtataacctctagtagcgatgaaagacatgaagagactccaacgcgagacataatacaaatactaaaatgtagtaatgcaaacgcagctatgttggctaaatttaaagacctatttggtattagttttacagaacttattagaccatttaagagtgataaatccacatgtacagattggtgtgtggccgcatttggaatagccccaagtgtggcggaaagtttaaaaacattaattaaaccatattgtttatatgtacatatacaatgtttatcgtgttcatggggtatggtaattctagcattattacgatttaaatgtgcaaaaaacagaacaacaattgaaaaactattatcaaaattgctatgtatttcagctgcaagtatgctaatacaaccaccaaaattacgtagtaccccagctgcgttatattggtttaaaacagcaatgtcaaatattagtgaggttgatggagaaacaccagaatggattcaaagacaaacagtattacagcatagttttaatgatgcaatatttgacctatctgaaatggtacaatgggcatatgacaatgattttatagatgatagtgatatagcatataaatatgcacaattggcagaaactaatagtaatgcatgtgcttttttaaaaagtaattcgcaagctaaaattgtaaaagattgtgcaacaatgtgtagacattataaacgagctgaaaaaagagaaatgacaatgtcacagtggattaaaaggcgatgtgaaaaggtggacgatgacggtgactggagggacatagtacgatttttaagatatcaacaagtagattttctggcatttttatctgcactaaaaaattttttacatggtgtgcctaaaaaaaattgcatacttatatatggagcaccaaacacaggtaaatcattatttggaatgagtctaatgcatttcttacaaggagctattatatcctatgtaaattctaaaagccatttttggttgcagccattatatgatgccaaaatagctatgttagatgatgctacatcgccatgttgggcatatatagaccaatatttaagaaatgcactagatggaaatcctatttcattagatgtaaagcataaagcattagtgcaattaaaatgcccacctttacttattacatcaaatataaatgcaggcaaagatgacaggtggccatacttacatagcagggtagtggtctttacatttcacaatgaattcccatttgataaaaatggaaacccagtgtatgggcttaatgataaaaactggaaatcctttttctcaaggacgtggtgcagattaaatttgcacgaggaagaggacaaagaaaatgatggagacgctttcccagcgtttaagtgtgtgtcaggacaaaatactagaacattacgagactga

SEQ ID NO: 53 - DNA, 21 bp, artificial
  Region of HPV53 sequence targeted by the reverse primer HPV16/31/33/33n/35/39/52/56/58/66R
agacagcggttatggcaattc

SEQ ID NO: 54 - DNA, 21 bp, artificial
  Reverse complement of SEQ ID NO: 53
gaattgccataaccgctgtca

SEQ ID NO: 55 - DNA, 1943 bp, artificial
  HPV 39 nucleotides 928 - 2871 E1 gene sequence
tggccaatcgtgaaggtacagacggggatgggtcgggatgtaacggatggtttctagtacaggcaatagtagataaacaaacaggcgacacagtgtcggaggatgaggatgaaaatgcaacagatacaggttcagacttggcagactttattgatgattccacagatatttgtgtacaggcagagcgtgagacagcacaggtacttttacatatgcaagaggcccaaagggatgcacaagcagtgcgtgccttaaaacgaaagtatacagacagcagtggcgacactagaccgtatggaaaaaaagtaggcaggaataccaggggaacactacaggaaatttcattaaatgtaagcagtacgcaggcaacacaaacggtgtattccgtgccagacagcggatatggcaatatggaagtggaaacagctgaagtggaggaggtaactgtagcaactaatacaaatggggatgctgaaggggaacatggcggcagtgtacgggaggagtgcagtagtgtggatagtgctatagatagtgaaaaccaggatcccaaatctccaactgcacaaattaaattattgttacaatccaataacaaaaaggctgcaatgctaacacaatttaaagaaacatatggactatcctttactgacctggtacgtacgtttaaaagtgataaaacaacatgtacagactgggtggcagccatatttggagtacatccaactattgcagaaggatttaaaacattaatcaacaaatatgccttatatacacatatacaaagcttagacacaaaacaaggagtactaattttaatgctaataagatatacatgtggaaaaaatagggttactgtaggaaagggattaagtacattgttacatgttccagaaagttgtatgcttctggagcctcctaaactgcgcagccctgtagcagcactatattggtatcgcacaggtatatccaatattagtgtggtaacaggggatacgccagaatggatacaacgattaactgttatacaacatggaatagatgatagtgtatttgacctatcggacatggtacaatgggcatttgacaatgaatatactgatgaaagtgacatagcatttaattatgcaatgttagcagattgtaacagtaatgctgcagcctttttaaaaagtaactgccaggcaaaatatgtaaaagattgtgcaacaatgtgtaaacattacaagcgagcacaaaaaaggcaaatgtccatgtctcaatggataaaatttaggtgtagtaaatgtgatgaaggcggggactggagacccatagtacaattcttaagatatcaaggaatagaatttatatcctttttatgtgcattaaaggaatttttaaagggtactcccaaaaaaaactgtatagttatatatggacctgcgaatacaggaaagtcacatttttgtatgagccttatgcattttttacagggcacagttatttcatatgtaaactccaccagccacttttggctagaaccacttgcagatgcaaaactagcaatgttagatgatgcaaccggtacctgctggtcatatttcgataattatatgagaaatgcattagatgggtatgcaataagtttagataggaaatataaaagtttactacaaatgaaatgtccaccattattaataacctccaataccaatcctgtggaagacgataggtggccatatttacgtagtaggctaacagtgtttaaatttcctaatgcatttccatttgaccaaaacaggaatccagtgtacacaatcaatgataaaaactggaaatgtttttttgaaaagacttggtgcagattagacttgcagcaggacgaggatgaaggagacaatgatgaaaacactttcacaacgtttaaatgtgttacaggacaaaatactagaatactatga

SEQ ID NO: 56 - DNA, 1931 bp, artificial
  HPV 45 nucleotides 914 - 2845 E1 gene sequence
tggcggatccagaaggtaccgacggggagggaacggggtgtaatggctggttctttgtagaaacaattgtagagaaaaaaacaggggatgtaatatcagatgatgaggatgaaactgcaacagatacagggtcggatatggtagattttattgacacacaattatccatttgtgaacaggcagagcaagagacagcacaggcattgttccatgcgcaggaagttcagaatgatgcacaggtgttgcatcttttaaaacgaaagtttgcaggaggcagcaaggaaaacagtccattaggggagcagctaagtgtggatacggatctaagtccacggttacaagaaatttcattaaatagtgggcacaaaaaagcaaaacgacggttgtttacaatatcagatagtggctatggctgttctgaagtggaagctgcagagactcaggtaactgtaaacactaatgcggaaaatggcggcagtgtacatagtacacaaagtagtggtggggatagtagtgacaatgcagaaaatgtagatccgcattgcagtattacagaactaaaggagctattacaagcaagtaacaaaaaggctgcaatgctggcagtatttaaagacatatatgggctgtcatttacggatttggttagaaattttaaaagtgataaaacaacatgtacagattgggtaatggctatatttggagttaatccaacggtagcagaaggctttaaaacattaattaaaccagcaacgttatacgcccatatccaatgtttagattgtaaatggggagtattaatattagctttattaagatataaatgtggcaaaaatagactaactgttgcaaaaggcttaagcacattgttgcacgtacctgaaacatgtatgttaattgaaccaccaaaattgcgaagtagtgttgcagcattatactggtatagaacaggtatatccaatattagtgaagtaagtggagacacacctgagtggatacaaagactgacaattattcaacatggtattgacgatagtaattttgatttgtcagacatggtgcaatgggcatttgataatgaccttacagatgaaagtgatatggcatttcaatatgcccaattagcagactgcaacagtaatgcagctgcatttttaaaaagtaactgccaagccaaatatttaaaagattgtgctgtaatgtgtagacattataaaagagcacaaaaacgccaaatgaatatgtctcaatggattaaatatagatgttccaaaatagatgaaggtggggattggagacccatagtacaattcctaagatatcagggagtagaatttattagctttttaagggcactaaaggaatttcttaaaggaacaccaaaaaaaaattgtatactgttatatggacctgcaaatacaggaaaatcgtattttggaatgagttttatacatttcctacaaggtgcaataatatcatttgtaaattcaaacagccatttttggttagaaccgttagcagatactaaggtagccatgttggatgatgccacacacacgtgttggacatattttgataattatatgagaaatgcattagatggtaatcctataagtatagacagaaagcataaaccattattacagctaaaatgtcctccaatcctattaacatccaatattgatccagcaaaagataataaatggccatatttagaaagtagggtgacggtatttacatttccacatgcatttccatttgataaaaatggtaatccagtatatgaaataaatgataaaaattggaaatgtttttttgaaaggacatggtccagattagatttgcacgaggacgatgaagatgcagacaccgaaggaatccctttcggaacgtttaagtgcgttacaggacaaaatactagaccactatga

SEQ ID NO: 57 - DNA, 1800 bp, artificial
  HPV 51 nucleotides 841 - 2640 comprising the E1 gene sequence
ttgcccgtgttgtgcgaacaactagcaacggcgatggactgtgaaggtacagaggatgagggggcggggtgtaatgggtggttttttgttgaagcaatagtagaaaaaaaaacaggagataatgtttcggatgatgaggatgaaaatgcagatgatacaggatctgatttaataaactttatagatagtgaaactagtatttgcagtcaggcggaacaggagacagcacgggcgttgtttcaggcccaagaattacaggcaaacaaagaggctgtgcatcagttaaaacgaaagtttctagtcagcccgcgaagcagcccattaggagacattacaaatcaaaacaacacacacagccatagtcaggcaaacgagtcacaagttaaaaggagattactggacagttatccggacagcggatatggcaatacacaagtggaaactgtggaagcaacgttgcaggtagatgggcaacatggcggttcacagaacagtgtgtgtagtagcggggggggcagtgttatggatgtggaaacaacagaaagctgtgcaaatgtagaactaaacagtatatgtgaagtattaaaaagcagtaatgcaaaagcaacgttaatggcaaaatttaaagagttgtatggtattagttataatgagttggtacgggtgtttaaaagtgataaaacatgttgtatagattgggtttgtgcattgtttggcgtttccccaatggtagcagaaaatttaaaaacactaattaagccattttgcatgtactaccatatacaatgtttatcatgtgattggggcaccattgtattaatgctaattaggttttcatgtgcaaaaaacagaacaacaattgctaagtgtttaagtacattagtaaatatcccacaatcacaaatgtttatagaaccaccaaaattacgtagtacacctgtggcattatatttttatagaacaggcatatcaaacattagcaatacatatggagagacacctgaatggattacacgacaaacgcaactacaacatagttttgaggatagtacctttgaattatcacaaatggtgcaatgggcatttgaccatgaagtattagatgatagtgaaatagcatttcattatgcacaattagcagatatagatagtaatgctgcagcgtttttaaagagtaattgccaagcaaaatatgtaaaagattgtgggaccatggcacggcattacaaacgagcacaaagaaaatcattatctatgtcagcctggataaggtatagatgtgatagagcaaaggatggaggcaactggagagaaattgctaaatttttaagatatcaaggtgtaaactttatgtcctttattcaaatgtttaaacagtttttaaaaggaacaccaaaacacaattgcatagtcatatatggcccaccaaacacaggcaagtcattatttgcaatgagcctaatgaagtttatgcaagggtccattatttcatatgtaaactctggtagtcatttttggttacagccactagaggatgctaaaatagcattgttagatgatgctacgtatgggtgttggacatatattgatcagtatttaagaaactttttagatggtaatccatgtagtatagatagaaaacataggagtttaatacaattagtatgtccaccattactaataacgtcaaacataaatccacaagaggatgcaaacctaatgtatttacatacaagggtaacagtattaaagtttttaaatacatttccatttgataacaatgggaatgctgtgtataca

SEQ ID NO: 58 - DNA, 21 bp, artificial
  sequence to which the reverse primer for HPV 51 hybridises
actggacagttatccggacag

SEQ ID NO: 59 - DNA, 1943 bp, artificial
  HPV 52 nucleotides 864 - 2807 E1 gene sequence
tggaggaccctgaaggtacagagggcgaaagggagggatgtacaggctggtttgaagtaaaggcaataatagaaaaacaaacaggagataacatttcagaggacgaggatgaaaatgcatatgatagtggaacagatctaatagattttatagatgattcaaatataaataatgaacaggcagaacatgaggcagcccgggcattgtttaatgcacaggaaggggaggatgatttacatgctgtgtctgcagtaaaacgaaagtttacaagcagtccggaaagtgctgggcaagatggtgtagaaaaacatggtagtccgcgtgcaaaacacatttgtgtaaatacagagtgtgttttaccaaaacgcaaaccatgtcacgtagaagacagcggctatggcaatagtgaagtggaagcgcagcagatggcagaccaggtagacgggcaaaatggcgactggcaaagtaacagtagtcaatcaagtggggtgggggctagtaattcagatgtaagttgtactagtatagaggacaatgaggaaaatagtaatagaacgctaaaaagcatacaaaatattatgtgcgaaaatagcataaaaacaacggtattatttaaatttaaagaaacatatggtgttagctttatggaattagtaagaccatttaaaagtaatagaagtagttgtacagattggtgtattataggaatgggagtaacaccatcagttgcagaaggattaaaagtattaatacagccctatagcatatatgcccatttgcaatgtttaacatgtgacagaggcgtgcttatactgctgctaattaggtttaaatgtggaaaaaacagattaacagtgtccaaactaatgtcacagctgttaaatataccagaaacacatatggtaatagaaccaccaaaattacgaagtgctacctgtgcattatattggtatagaacaggtttgtctaatattagtgaggtatatggtaccaccccagaatggatagaacaacaaacagtattacagcatagctttgacaatagcatatttgattttggagaaatggtgcaatgggcatatgatcatgatataacagatgatagtgacatagcatataaatatgcacagttagcagatgtaaatagcaatgctgcagcattcctaaaaagcaattcgcaagcaaaaatagtaaaggactgtgcaaccatgtgtagacattataaacgggcagaaagaaaacatatgaatattggacaatggatacagtatagatgtgatagaatagatgatggtggagattggaggcctatagtaagatttttaagatatcaagacatagaatttacagcctttttagacgcatttaaaaaatttttaaaaggtatacctaaaaaaaattgtttagtattatatggacctgcaaacacaggaaaatcatattttggaatgagtttaattaggttcttaagtggatgtgtaatatcctatgtaaactcaaaaagccatttttggctacaaccattaacagatgcaaaagtgggtatgatagatgatgtaacacctatatgttggacatatatagatgattatatgagaaatgcactggatggaaatgatatatcagtagatgtaaagcatagagccttagtacaaataaaatgcccaccattaattttaacaacaaatacaaatgcaggaacagatcctaggtggccatatttacatagtagattggttgtgtttcatttcaaaaacccatttccatttgatgaaaatggcaatcccatatatgaaattaacaacgaaaattggaaatcctttttctcaaggacgtggtgcaaattagatttaatacaggaagaggacaaggaaaacgatggagtcgataccggcacgtttaaatgcagtgcaggaaaaaatactagatctatacgaagctga

SEQ ID NO: 60 - DNA, 21 bp, artificial
  Region of HPV 52 that is targeted by the reverse primer
agacagcggctatggcaatag

SEQ ID NO: 61 - DNA, 21 bp, artificial
  the reverse complement of SEQ ID NO: 60
ctattgccatagccgctgtct

SEQ ID NO: 62 - DNA, 1911 bp, artificial
  HPV 56 nucleotides 895 - 2805 E1 gene sequence
atggcgtcacctgaaggtacagatggggaggggaagggatgttgtggatggtttgaagtagaggcaattgtagaaaaaaaaacaggagataaaatatcagatgatgaaagtgacgaggaggatgaaatagatacagatttagatggatttatagacgattcatatatacaaaatatacaggcagacagagagacagctcaacaattgttgcaagtacaaacagcacatgcagataaacagacgttgcaaaaactaaaacgaaagtatatagctagtccattaagtgatattagtaatcagcaaactgtgtgccgggaaggagtaaaacggaggcttattttatcagacctacaagacagcgggtatggcaatacattggaaactctggaaacaccagaacaggtagatgaagaggtacagggacgtgggtgcgggaatacacaaaatggaggctcacaaaacagtacctatagtaacaatagtgaggactctgtaatacatatggatattgatagaaacaatgaaacgccaacacaacaattgcaggacttgtttaaaagtagcaatttacaaggtaaattatattataaatttaaagaagtgtatggtattccattttcagaattggtgcgtacgtttaaaagtgatagtacatgttgcaatgattggatatgtgctatatttggtgttaatgaaacattagccgaggcactaaaaactataataaaaccacactgtatgtattatcatatgcaatgtttaacatgtacatggggggttatagtaatgatgctaattagatatacatgtggcaaaaacagaaaaacaattgcaaaagcattaagctcaatattaaatgtaccacaggagcaaatgttaattcaaccaccaaaaatacgaagtcctgctgtagctttatatttttataaaacagcaatgtcaaatattagtgatgtgtatggagacacaccagaatggatacaaagacaaacacaattgcaacacagtttacaggatagtcaatttgaattatctaaaatggtgcagtgggcatttgataatgaagtaacagatgatagccaaattgcgtttcaatatgcacaattagcagatgtagacagcaatgcacaagcctttttaaaaagcaatatgcaggcaaaatatgtaaaggattgtggaataatgtgtagacattataaaagggcacaacagcaacaaatgaatatgtgccagtggataaagcacatatgtagtaaaacagatgaagggggtgattggaaacccattgtacaatttttaagatatcaaggggtcgatttcatttcatttctaagttactttaaattatttctacaaggaacacctaaacataactgtttggtactttgtggaccgccaaatacaggtaaatcatgctttgctatgagtcttataaagttttttcaagggtctgtcatttcatttgtgaattcacaaagccacttttggttgcagccattagacaatgctaaacttgggttgttggatgatgcaacagaaatatgttggaaatatatagacgattatttaaggaatttggtagatggaaatcctataagtttagatagaaaacataaacaattagtacaaataaaatgtccaccattactaattacaaccaatataaatcctatgctagatgctaaattacgatatttacacagtagaatgttagtgtttcagtttcaaaatccatttccattagataataatggtaatcctgtatatgaattaagtaatgtaaactggaaatgtttctttacaaggacgtggtccagattaaatttggataacgacgaggacaaagaaaacaatggagacgctttcccaacgtttaaatgtgtgccagaacaaaatactagactgttttga

SEQ ID NO: 63 - DNA, 1893 bp, artificial
  HPV 66 nucleotides 895 - 2787 E1 gene sequence
atggcatcacctgaaggtacaaatggggaggggacgggatgttgtggatggtttcaggtagaagcaattgtagaaagaaaaacgggggatacaatatcagatgatgaaagcgaggaggagaatgaaacagatacagatttagatggatttatagacaatacacttataaacaatacacaggaagacagggagacagctcaacaattattgcaagtacaaacagcacatgcagatgcacagacgttgcaaaaactaaaacgaaagtatataggtagtcccttaagtgatattagtaatcagcaaactgtgtaccgggaggaagtaaaacgaaggctaatattatcagaagacagtgggtatggcaatacattggaaacattggaaacatcacaacaggtagaatacgaaaagggaaatgggtgcgggagctcacaaaatggaggctcgcaaaacagtaactgtagtgagcactcggtggtaaatatggatatagatacaaatatggaaacaccaacacaccaattgcaggaactatttaaaagtagtaacgcacaaggaagattacattttaaatttaaagaagtgtatggagtgccatatacagagttggtgcgaacatttaaaagcgatagtacatgttgtaacgattggatatgtgcaatatttggtgttaatgaaacattagcagaagcgttaaaaactatactaaaaccacaatgtgtgtactatcatatgcaatgcctaacatgttcatggggggtaattgtaatgatgctaattagatatatatgtggaaaaaacagaaaaacaattacaaaatcgctaagctcaattttaaatgtaccacaagagcaaatgttaattcaaccaccaaaactacgaagtcctgctgtggcattatatttttataaaacagcaatgtcaaatattagtgaggtgtatggggaaacaccagaatggatacaaagacagacacaattgcaacacagtttacaagacaatcaatttgaattgtctaaaatggtacagtgggcatttgataatgacgtaacagatgatagccaaattgcctttttatatgcacaactagcggacatagatagtaatgcacaagcatttttagaaagtaatatgcaagcaaaatatgtaaaggattgtggaataatgtgtagacattataaaagggcacagcaacagcaaatgaatatgtgccagtggataaagcatatatgtagtaaagtagatgaagggggtgattggaaacccattgcgcaatttttacgatatcaaggggtcgacttcatttcatttttaagttattttaaattatttttacaaggcacgcctaaacataattgtttggtactgtgtggaccaccaaatacaggtaaatcatgttttgctatgagccttataaattttttccaagggtcagtcatttcatttgttaattcacaaagccacttttggttacagccactagacaatgccaaattagggttgctggatgatgcaacagatacgtgttggagatatatagatgattatctaagaaatttattagatgggaatcccataagtttagataggaaacataaacaattagtacaaataaaatgtcctccagtcattattacaactaatgtaaatcctatgcaagatgcaaaattaagatatctacacagtagaatttcagtgtttaagtttgaaaatccatttccattagataacaatggtaatcctgtgtatgaattgagtaatgtaaattggaaatgtttttttgaaaggacatggtccagattaaattcggataacgacgaggacaaagaaaacaatggagactctatcccaacgtttagatgcgtgccagaacaaaatactaggctgttatga

SEQ ID NO: 64 - DNA, 21 bp, artificial
  target sequence of HPV 66 for the reverse primer. HPV16/31/33/33N/35/39/52/56/58/66R
agacagtgggtatggcaatac

SEQ ID NO: 65 - DNA, 21 bp, artificial
  reverse complement of SEQ ID NO: 64
gtattgccatacccactgtct

SEQ ID NO: 66 - DNA, 1935 bp, artificial
  HPV 58 nucleotides 883 - 2817 E1 gene sequence
atggatgaccctgaaggtacaaacggggtaggggcgggctgtactggctggtttgaggtagaagcagtaatagaacgaagaacaggagataatatttcagatgatgaggacgaaacagcagacgatagtggtacagatttaatagagtttatagatgattcagtacaaagtactacacaggcagaagcagaggcagcccgagcgttgtttaatgtacaggaaggggtggacgatataaatgctgtgtgtgcactaaaacgaaagtttgcagcatgctcagaaagtgctgtagaggactgtgtggaccgggccgcaaatgtgtgtgtatcgtggaaatataaaaataaagaatgcacacacagaaaacgaaaaattattgagctagaagacagcggatatggcaatactgaagtggaaactgagcagatggcacaccaggtagaaagccaaaatggcgacgcagacttaaatgactcgcagtctagtggggtgggggctagttcagatgtaagcagtgaaacggatgtagacagttgtaatactgttccattacaaaatattagtaatattttacataacagtaatactaaagcaacgctattatataaattcaaagaagcttatggagtaagttttatggaattagttagaccatttaaaagtgataaaacaagctgtacagattggtgtataacagggtatggaataagtccctccgtagcagaaagtttaaaagtactaattaaacagcacagtatatatacacacctacaatgtttaacgtgtgacagaggaattatattattattgttaattagatttaaatgtagcaaaaatagattaactgtggcaaaattaatgagtaatttactatcaattcctgaaacatgtatgattattgagccaccaaaattacgaagtcaagcatgtgccttatattggtttagaacagcaatgtcaaatataagtgatgtgcaagggacaacaccagaatggatagatagattaacagtgttacagcatagctttaatgatgatatatttgatttaagtgaaatgatacaatgggcatatgataatgacattacagatgatagtgacattgcatataaatatgcacagttagcagatgttaatagtaatgcagcagcatttttaagaagcaatgcacaagcaaaaatagtaaaagactgtggcgttatgtgcagacattataaaagagcagaaaagcgtggtatgacaatgggacaatggatacaaagtaggtgtgaaaaaacaaatgatggaggtaattggagaccaatagtacaatttttaagatatcaaaatattgaatttacagcatttttagttgcatttaaacagtttttacaaggtgtaccaaaaaaaagttgtatgttactgtgtggcccagcaaatacagggaaatcatattttggaatgagtttaatacatttcttaaaaggatgcattatttcatatgtaaattccaaaagtcatttttggttgcagccattatcagatgctaaactaggtatgatagatgatgtaacagccataagctggacatatatagatgattatatgagaaatgcattagatggtaacgacatttcaatagatgtaaaacatagggcattagtacaattaaaatgtccaccattaataattacctcaaatacaaatgcaggcaaagattcacgatggccatatttgcacagtagactaacagtatttgaatttaacaatccatttccatttgatgcaaatggtaatccagtgtataaaataaatgatgaaaattggaaatcctttttctcaaggacgtggtgcaaattaggcttaatagaggaagaggacaaggaaaacgatggaggaaatatcagcacgtttaagtgcagtgcaggacaaaatcctagacatatacgaagctga

SEQ ID NO: 67 - DNA, 1934 bp, artificial
  HPV 59 nucleotides 872 - 2806 E1 gene sequence
tggccgattcggaaggtacagatggggaagggacggggtgcaatggatggttttttgtgcaggcaatagtagataaaaaaacaggtgacaaaatttcagatgacgaggatgaaaatgcaacagatacaggttcagacttggtagactttattgatgataccacaacaatttgtgtacaggcagagcgcgagacagcacaggccttgtttaatgtgcaggaagcccaaagggatgcacgggaaatgcatgttttaaaacgaaagtttgggtgcagtatagaaaacagtagtgagaaagcggcggcaggaacaaaagctaagtcaccattacaagaaatatcagtaaatgttaaccacccaaaagtaaagagaaggttactaacagtgccagacagcggctatggctattctgaagtggaaatgctcgagactcaggtaaccgtggagaatactggaaatggggacagcaatggcagtgtttgtagcgacagtcaaatagacggtagcgacagcagtaacatggatgttgaaaacatagttccaacatcccccactaatcaattgttacagttattacatagcaaaaataagaaagcagctatgtatgcaaaatttaaagaattgtatgggttatcatttcaagatttggttaggacatttaaaagtgacagaactacctgtagcgattgggtaaccgccatttttggtgtaaatccaactgtagcagaaggatttaaaacattaatacaaccctatgtgctatatgcacatatacaatgtttagattgtgcatggggagtagtaatattagcattattaagatataaatgtggaaaaaatagaataacagttgcaaaaggacttagcacattactacatgtaccagatacgtgcatgttaattgaaccacccaaattgcgtagtggtgttgcagcattatattggtacagaacaggaatgtccaatattagtgaagttataggggaaacgcccgaatggatacaaagactaacaattatacaacatggagttgatgatagcgtgtttgacctgtcagaaatgatacaatgggcgtttgataatgacctaacagatgaaagtgatattgcatatgaatatgcattaatagcagatagtaatagtaacgccgctgcatttttaaaaagcaactgccaggcaaaatacctaaaagattgtgcagttatgtgtaggcattataaaagagcacaaaaaagacaaatgagtatgtcacagtggataaaatggagatgtgataaaatagaagaggggggagattggaaacccatagtacaatttttaagatatcaaggagtagaatttataacgtttttatgtgcattaaaagattttttaaaaggtaccccaaaaagaaattgcattgtgctgtgtgggccagcaaatacaggcaagtcatactttggaatgagcctgctacatttcttacaaggaactgtaatttcacatgtaaattcaaatagtcacttttggctagaacctttaacagatgctaaattagctatgctagacgatgcaacagatagttgttggacatattttgatacatatatgcgaaatgctctggatggcaatcctataagtgtagatagaaagcataggcacctagtacaaattaaatgtccaccaatgcttattacatcaaatacaaatccagttacagataacaggtggccatacctaaatagcagactaatggtatttaaatttccaaacaaatttccatttgacaaaaatagaaatccagtatatacaattaatgacagaaactggaaatgtttttttgaaaggacgtggtgcagattagatttgaacgaggaagaggatgatgcagacagtgatggacaccctttcgcagcgtttaagtgtgttacaggatcaaatattagaacattatga

SEQ ID NO: 68 - DNA, 24 bp, artificial
  region targeted by the reverse primer for HPV 59, m denotes a nucleotide change c/a
gaaggttamtaacagtgccagaca

SEQ ID NO: 69 - DNA, 1923 bp, artificial
  HPV 68 nucleotides 824 - 2746 E1 gene sequence
atggccaattgtgaaggtacagatggggacgggacggggtgtaacggatggtttttcgtacaagcaatagtagataaacaaacaggtgacacagtctcagaggatgaggatgaaaacgcgacagatacaggttcagacatggtagatttcattgatgatgctacagatatttgtatacaggcagagcgtgagacagcacaggtactgttaaatatgcaacaggcccaaagggatgcacaaacagtgcgtgccctaaaacgaaagtatacagacagtatagaaagcagccctttagcaaagtcgccattacaggaactatcaataaatgtaagcagtacacaggcaagacaaccggcgtatacagtgccggacagcggctatggcaatatggaagtggaaactcactcggaggtaactgtagcaactaatacaaatggggcggacggggaggatgaaggggaaaatggcgacagcatacgggaggactgtagtagtgtagacagtgctatagatagtgaaaaccaggatcctaaatcacctactactcaactaaaagtattattacaatgtaataataaaaaagctgcaatgttaacagaatttaaaaaagtatatggattgtcctttaatgacctagtacgtacatttaaaagtgataagaccacatgtacggactgggtagcagcaatatttggagtaaatccaaccattgccgaagggtttaaaacactaattaaacaatatgcattatatacccatatacaatgtttagatacaaaaaacggaatattaatattaatgttaataagatataaatgtgggaaaaatagaataacagtaggaaaaggattaagtacattgttgcatgttccagacagctgtatgcttttgcagccaccaaaattgcgtagccctgttgcagcattgtattggtatagaacaggaatatctaatattagtgaggtgtgtggagacacgccagaatggataaaaagattaactataatacaacatggaatagatgatagtgtatttgatctatcagacatggtacaatgggcatttgataatgagttaacagatgaaagtgatatagcattttcatatgctatgttggcagattgtaatagtaatgctgcagcgtttttaaaaagcaactgccaagcaaaatatgtaaaagattgtgcaacaatgtgtagacattacaaacgggcacaaaaacgacaaatgtcaatgccgcaatggattaaattcagatgcagtaaatgtgatgaaggtggtgattggcgaccaattgtacagtttttaagatatcaggggctagaattcataacattcttatgtgcattaaaagattttttaaaaggcacgccaaaacgtaattgtatagttatacatgggccaccaaatactggcaagtcatatttttgcatgagtcttatacatttcttgcaaggcacaataatttcatatgtaaattcagctagtcacttttggttagagccacttgcagatgcaaaaatagccatgttagatgacgcaacaggtacatgttggtcatattttgataattacatgagaaatgcattagatggtaacccaataagtttagatagaaaacacagacacctaatacaaataaaatgtccaccaatgctaataacatcaaatactaaccctgtggaagacaataggtggccgtatttacatagcagactaactgtatttaaatttcctaatgcatttccatttgaccaaaacaggaacccagtgtatacaatgaatgataaaaactggaaatgtttttttgaaaagacttggtgcaaattagacttgcagcaggacgaggatgaaggagacaatgatgaaaacactttcccaacgtttaaatgtgttacaggagaaaatattagaacattatga

SEQ ID NO: 70 - DNA, 21 bp, artificial
  region of HPV68 to which the reverse primer for HPV68 hybridises
agcaaagtcgccattacagga

SEQ ID NO: 71 - DNA, 24 bp, artificial
  Target sequence for HPV 16 R HEX
ataatgattatttaacacaggcag

SEQ ID NO: 72 - DNA, 21 bp, artificial
  Target sequence for HPV18 R HEX
aaggaacattttgtgaacagg

(19) United States
(12) Patent Application Publication
(10) Pub. No.: US 2017/0029908 A1
(43) Pub. Date: Feb. 2, 2017

(54) SCREENING TEST FOR DETECTING THE PRESENCE OF ONCOGENIC HPV VIRUSES

(71) Applicants: CERVICO SP. Z O.O, Szczecin (PL); CENTRUM ONKOLOGII-INSTYTUT IM. MARII SKLODOWSKIEJ- CURIE, Warszawa (PL)

(72) Inventors: Jacek PODOLSKI, Szczecin (PL); Lucjan WYRWICZ, Warszawa (PL)

(21) Appl. No.: 15/303,503

(22) PCT Filed: Apr. 11, 2015
(86) PCT No.: PCT/PL2015/050007

§ 371 Date: Oct. 11, 2016

(30) Foreign Application Priority Data
Apr. 11, 2014 (PL) P.407864

(51) Int. Cl. C12Q 1/70

(52) U.S. Cl. CPC C12Q 1/708; C12Q 2600/158; C12Q 2600/16

(57) ABSTRACT

The screening test and process for detecting the presence of oncogenic HPV viruses, in particular based on LNA (locked nucleic acids) technology, are effective tools in the prevention and early detection of cervical cancer. Probes for use in HPV detection by real time PCR target a conserved region of oncogenic HPV genomes.

### CROSS-REFERENCE TO RELATED APPLICATIONS

See also Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

1. Field of the Invention

The invention relates to the field of diagnostics of cervical cancer, and in particular to a screening test for oncogenic HPV viruses, preferably based on LNA (locked nucleic acids) technology and an associated process, as a new tool in prevention and the early detection of cervical cancer.

2. Description of Related Art Including Information Disclosed Under 37 CFR 1.97 and 37 CFR 1.98.

Cervical cancer is a malignant tumour, which is still a major health and social problem worldwide. Every year approximately 500,000 new cases of this disease are diagnosed in the world, and more than half of the women diagnosed die of the disease. Although all women are vulnerable to this type of cancer, significant geographical differences of incidence and mortality have been noted.

Almost 80% of cases of cervical cancer are diagnosed in developing countries, in which this disease can even place first as a leading cause of death among women in comparison with other genital cancers. In these countries, an increase in the number of cases is expected in the coming years. Downward trends in incidence of cervical cancer and a lower mortality rate in developed countries are associated with the introduction of general and systematic prevention and better access to modem health care.

Poland belongs to those countries with a relatively high incidence and mortality rate of cervical cancer. It is the second most common malignant tumour in women up to 45 years of age. Every year approximately 3,500 Polish women are diagnosed with cervical cancer, and half of them die from this. The number of deaths of this type of cancer places Poland on one of the top positions in Europe.

The treatment of cervical cancer depends on the stage of the disease and comprises a local excision and radical surgery in early stages of the disease, radical radiotherapy in combination with brachytherapy and concurrent chemotherapy in advanced stages and palliative care in case of metastatic disease. Carboplatin and paclitaxel are most commonly used within chemotherapy treatment. Additional drugs may increase the survival time of patients in advanced cases. Recent reports suggest the possibility of the additional application of targeted therapy (bevacizumab) for this indication, which improves the parameters of life expectancy in palliative care; however, it significantly affects the cost of such treatment. In case of resistance to platinum, the second drug of choice is topotecan.

In 2006, vaccines aimed at preventing HPV infections, the major cause of incidences of cervical cancer, were placed on the market. A vaccine called Gardasil has been approved for use in the United States and within the European Union. Another vaccine against HPV infections is Cervarix.

Cervical cancer is one of the few malignant neoplasms, which, however, can be effectively prevented by proper prophylaxis and early diagnostics of medical conditions that are predisposed to its development (i.e. precancerous lesions, known as intraepithelial neoplasia). Cytological examinations of cervical smears aimed at detecting abnormal cells of stratified squamous or glandular epithelium regularly performed in women have been, so far, the basic and most effective method of such prophylaxis. Despite the undoubted value of cytological examination in a population-based screening for cervical cancer, the basic limitation of this method is its relatively low sensitivity of approx. 60%. According to many scientific studies, a combined cytological and molecular genetic examination for so-called oncogenic types of human papillomavirus (HPV), a group of 15 out of hundreds of known types of HPV, which directly cause the process of carcinogenesis in the epithelium of the cervix in some of the infected women (otherwise referred to as high-risk viruses, high-risk HPV, HR-HPV), is much more effective in detecting advanced precancerous lesions (so-called CIN2, CIN3, carcinoma in situ). In addition to the high sensitivity of a combined cytological and HPV examination (up to 100%), and only slightly lower specificity in comparison to cytological examination alone, the method is also characterised by a high so-called NPV (negative predictive value) index. Such combined examinations are especially recommended for women over 30 years of age. In addition to the use of HPV tests in primary screening for cervical cancer, as a supplement to cytological diagnostics, the determination of viral infections caused by the oncogenic HPV group is also recommended at the stage of so-called enhanced diagnostics in women with a detected presence of abnormal epithelial cells, especially in the case of diagnoses known as ASC-US and LSIL (recommendations of the Polish Gynaecological Society and international institutions). Execution of the test for the presence of oncogenic HPV types in women with certain abnormalities in a cytological preparation allows for a more specific further diagnosis and possible treatment.

Therefore, the common use of molecular genetic tests to detect oncogenic HPV types, as a supplement to population-based screening programmes based on cytological examinations of cervical smears, seems reasonable. Unfortunately, due to technical reasons (high complexity of tests, in which several dozen HPV types with low genome homology are analysed in a single assay) and the cost of commercially available HPV tests, such combined preventive exams are not common within population-based screening programmes, both in Poland and around the world, and they are limited exclusively to the stage of enhanced diagnostics in the case of detection of abnormal cells in cytological screening tests. Another problem is the lack of a uniform methodical standard for HPV tests.

The Hybrid-Capture (Digene) method is considered the gold standard for HPV diagnostics. However, this method has relatively low diagnostic sensitivity. In addition, the test allows for the detection of only some of the known oncogenic HPV types. Most of the tests available on the HPV diagnostics market are based on genotyping (determination of individual HPV types) methods, which generate high cost as well as time and labour consuming analyses and a reduction of specificity of the method for the detection of morphological abnormalities of the cervix. In recent years, a few tests approved for clinical use (CE-IVD-certified) based on different molecular genetic methods, using DNA amplification techniques (e.g., PCR) for detection of infections with HPV of the group characterised by a high-risk of cervical cancer development (with no distinction of type), appeared on the market. However, in general, they do not comprise an analysis of all oncogenic HPV types, and their high price prevents their common use in population-based screening tests of cervical cancer in Poland.

The PCR method in real time (real-time PCR) is now widely used in viral diagnostics, but it requires an individual approach for each investigated microorganism. To use this method for diagnostics, it is necessary to adequately select sequences, so-called primers and molecular probes, as well as reaction conditions, in order to ensure the highest clinical credibility of the test and, in particular, an adequate threshold of pathogen detection and diagnostic specificity. Preferably, the multiplex reaction allows for single-stage diagnosis of multiple HPV types, which is however an extremely difficult task and requires one to obtain a test characterised by a high sensitivity and specificity of detection.

Real-Time PCR assays in virology were reviewed by Mackay et al., 2002, Nucleic Acid Research, 30(6), 1292-1305. WO08/145366 (Univ. Milano Bicocca) described a method of identifying and quantifying oncogenic HPV nucleic acids. The method used 5 independent real-time PCR assays as a first line screen to identify the presence of HPV in a sample, and further TaqMan real-time PR assays performed on positive samples to determine the presence and the viral load of the most common oncogenic HPV types. In Quantitative Real-Time PCR: Methods and Protocols, Chapter 8 in Methods in Molecular Biology, vol. 1160:87-97 2014, Broccolo described a multiplex real-time PCR platform integrated into an automated extraction method for the rapid detection and measurement of oncogenic HPV DNA load from cervical samples. In this system, two multiplex qPCR assays (first line screening) were performed to detect and quantify HPV-16, -18, -31, -33, -45, -52, and -58 (HPV-18 and -45 measured together by single-fluorophore). In addition, a multiplex qPCR assay specific for HPV-18 and HPV-45 was used to type precisely the samples found to be positive for one of the two strains. Micalessi et al. described a TaqMan-based quantitative real time PCR assay for high-throughput detection of 17 HPV genotypes in seven multiplex reactions, and reported an analytical sensitivity of <100 copies (Clin. Chem. Lab. Med., 50(4):655-661 2011).

There is great demand for performing a test for the presence of oncogenic HPV types in order to implement the appropriate diagnostic and therapeutic procedure. The number of HPV tests performed is definitely not sufficient due to their high costs and limited availability on the market. The development of an inexpensive and clinically credible test for the presence of oncogenic HPV types will significantly improve the availability of modern diagnostics of medical conditions predisposing to cervical cancer.

Therefore, the aim of the invention is to develop a diagnostic test which would allow for sensitive, specific and inexpensive detection of oncogenic HPV types in a cervical smear collected on the liquid medium (liquid based cytology—LBC).

### BRIEF SUMMARY OF THE INVENTION

The invention provides a method of determining if a sample tests positive for oncogenic human papillomavirus (HPV), the method comprising:
combining the sample with primers for real-time polymerase chain reaction (PCR) amplification of a target region of oncogenic HPV genomes, and a labelled pan-HPV probe capable of emitting a signal upon amplification of the target region,
providing conditions for real-time PCR, and
measuring for a signal emitted from the labelled pan-HPV probe,
wherein detection of the signal indicates that the sample tests positive for oncogenic HPV,
characterised in that the pan-HPV probe is a labelled locked nucleic acid (LNA) molecule comprising sequence ACTTTCGTTT (SEQ ID NO: 25) or the reverse complement thereof.

Preferably, the pan-HPV probe is a labelled 10-nucleotide molecule of sequence consisting of ACTTTCGTTT (SEQ ID NO: 25) or the reverse complement thereof.

Preferably, the pan-HPV probe is labelled at its 5′ end with a fluorophore and at its 3′ end with a quencher, and wherein the signal emitted upon amplification of the target region is fluorescence.

Preferably, the primers are for amplification of a target region of any of HPV16, HPV18, HPV31, HPV33, HPV35, HPV39, HPV45, HPV51, HPV52, HPV56, HPV58, HPV59, HPV66 or HPV68.

Preferred methods of the invention comprise combining the sample with a combination of primers for amplification of a target region of all of HPV16, HPV18, HPV31, HPV33, HPV35, HPV39, HPV45, HPV51, HPV52, HPV56, HPV58, HPV59, HPV66 or HPV68. The primers may comprise one or more of the following primer pairs:

a) for HPV16 SEQ ID NO: 1 and SEQ ID NO: 14

b) for HPV18 SEQ ID NO: 2 and SEQ ID NO: 15

c) for HPV33/33n SEQ ID NO: 3 and SEQ ID NO: 14

d) for HPV31 SEQ ID NO: 4 and SEQ ID NO: 14

e) for HPV35 SEQ ID NO: 5 and SEQ ID NO: 14

f) for HPV39 SEQ ID NO: 6 and SEQ ID NO: 14

g) for HPV45 SEQ ID NO: 7 and SEQ ID NO: 15

h) for HPV51 SEQ ID NO: 8 and SEQ ID NO: 16

i) for HPV52 SEQ ID NO: 9 and SEQ ID NO: 14

j) for HPV56/66 SEQ ID NO: 10 and SEQ ID NO: 14

k) for HPV58 SEQ ID NO: 11 and SEQ ID NO: 14

l) for HPV59 SEQ ID NO: 12 and SEQ ID NO: 17

m) for HPV68 SEQ ID NO: 13 and SEQ ID NO: 18

In a further preferred method, wherein the primers comprise all of the primer pairs (a) to (m), a method in accordance with the invention may comprise:
combining the sample with primers for amplification of a target region of HPV16, HPV18, HPV31, HPV33, HPV35, HPV39, HPV45, HPV51, HPV52, HPV56, HPV58, HPV59, HPV66 and HPV68,
detecting a signal from the labelled pan-HPV probe, and
concluding that the sample tests positive for HPV16, HPV18, HPV31, HPV33, HPV35, HPV39, HPV45, HPV51, HPV52, HPV56, HPV58, HPV59, HPV66 or HPV68.

A method in accordance with the invention may comprise:
combining the sample with primers for amplification of a target region of HPV16, HPV18, HPV31, HPV33, HPV35, HPV39, HPV45, HPV51, HPV52, HPV56, HPV58, HPV59, HPV66 and HPV68,
detecting no signal from the labelled pan-HPV probe, and
concluding that the sample tests negative for HPV16, HPV18, HPV31, HPV33, HPV35, HPV39, HPV45, HPV51, HPV52, HPV56, HPV58, HPV59, HPV66 and HPV68.

A method according to the invention may further comprise:
combining the sample with primers for amplification of a target region of HPV33n, and a labelled 33n probe capable of emitting a signal upon amplification of the target region, wherein the 33n probe and the pan-HPV probe carry the same label,
providing conditions for real time-PCR, and
measuring for a signal emitted from the labelled 33n probe and/or the pan-HPV probe,
wherein detection of the signal indicates that the sample tests positive for oncogenic HPV,
characterised in that the 33n probe is a labelled locked nucleic acid (LNA) molecule comprising sequence ACTTTCGCTT (SEQ ID NO: 26) or the reverse complement thereof. In such methods the 33n probe may be a labelled 10-nucleotide molecule of sequence consisting of ACTTTCGCTT (SEQ ID NO: 26) or the reverse complement thereof.

The pan-HPV probe and 33n probe may be labelled at the 5′ end with a fluorophore and at the 3′ end with a quencher, and wherein the signal emitted upon amplification of their respective target regions is fluorescence of the same wavelength.

A method according to the invention, may comprise determining whether the sample tests positive for HPV16 or HPV18, the method comprising
combining the sample with primers for real-time PCR amplification of a target region of HPV16 and HPV18 genomes, and a labelled 16/18 probe capable of emitting a signal upon amplification of the target region,
providing conditions for real time PCR, and
measuring for a signal from the labelled 16/18 probe,
wherein detection of a signal indicates that the sample tests positive for HPV16 or HPV18,
characterised in that the 16/18 probe is a labelled locked nucleic acid (LNA) molecule comprising sequence ATATCWGATGACGAG (SEQ ID NO: 27) or the reverse complement thereof.

The 16/18 probe may be a labelled 15-nucleotide molecule of sequence consisting of ATATCWGATGACGAG (SEQ ID NO: 27) or the reverse complement thereof.

The 16/18 probe may carry a different label from the pan-HP V probe.

The 16/18 probe may be labelled at its 5′ end with a fluorophore and at its 3′ end with a quencher, and wherein the signal emitted upon amplification of the target region is fluorescence.

The 16/18 probe may emit a fluorescent signal at a wavelength different from that of the pan-HPV probe.

The primers for use in a method according to the invention for determining whether the sample tests positive for HPV16 or HPV18 may comprise one or both of the following primer pairs:

a) for HPV16 SEQ ID NO: 19 and SEQ ID NO: 20
b) for HPV18 SEQ ID NO: 21 and SEQ ID NO: 22.

A method according to the invention may further comprise conducting a positive control for the real-time PCR, comprising
combining the sample with primers for real-time PCR amplification of a control DNA sequence present in the sample, and a labelled control probe capable of emitting a signal upon amplification of the control DNA sequence,
providing conditions for real-time PCR, and
measuring for a signal from the control probe,
wherein detection of the signal indicates that the real-time PCR is functional.

The control DNA sequence may be a human DNA sequence.

In a method according to the invention the sample may be a liquid-based cytology sample from a human cervical smear.

The invention further provides a composition comprising a labelled nucleic acid probe comprising nucleotide sequence ACTTTCGTTT (SEQ ID NO: 25) or the reverse complement thereof, and a detectable label. The probe may comprise LNA nucleotides. Preferably, the probe is a 10-nucleotide molecule of sequence consisting of ACTTTCGTTT (SEQ ID NO: 25) or the reverse complement thereof, labelled with a fluorophore and a quencher. The probe may be labelled at its 5′ end with a fluorophore and at its 3′ end with a quencher.

The invention additionally provides the use of a composition according to the invention in performing real-time PCR for detection of HPV.

Surprisingly, the inventors realised the above objective to obtain a diagnostic test for the presence of oncogenic HPVs, characterised by many advantages, such as ease of implementation, low cost, favourable parameters: high sensitivity, clinical effectiveness, specificity, and a clinically desired detection threshold for HPV16/18 and other oncogenic HPV types. Moreover, as demonstrated below, this test can potentially allow for resignation from a cytological examination in a large population of HR-HP V-negative women.

The HPV genome has approximately 8 thousand nucleotides, and the length of continuous sequences with full homology between oncogenic types does not exceed several dozen base pairs. Such great diversity of HPVs does not allow for the design of a minimal amount of hybridisation probes, which would enable the detection of all oncogenic HPV viruses in one reaction. This problem can be solved using modified LNA (locked nucleic acids) nucleotides in a molecular probe. LNA nucleotides contain an additional methylene bridge within the molecule of deoxyribose, which, due to the change of conformation and rigidifying the molecule, increases the melting point of oligomers built from such nucleotides and the stability of the bonds between complementary nucleotides in the hybridisation process. Properties of LNA were reviewed by Vester & Wengel, Biochemistry 43(42): 13233-13241 2004. According to the invention, thanks to the use of LNA, it is possible to advantageously apply significantly shorter, in comparison to traditional ones, TaqMan type molecular probes, with a high specificity of hybridisation into the PCR amplification product at a temperature optimal for the reaction, in a PCR. The use of significantly shorter 8-10 nucleotide long LNA-TaqMan probes (in comparison to 20-30 nucleotides in traditional probes) allows for their easier fitting into short (of even 10-12 base pairs) homologous regions between different oncogenic HPV types and consequently for the development of a new diagnostic test.

There is a high demand for tests using LNA technology, which would allow for obtaining clinically relevant expertise within the field of prevention and diagnostics of cervical cancer. The patients also want to be subjected to modern preventive exams that would be highly sensitive and financially available.

Accordingly, a test called HR-HPV, preferably based on a multiplex real-time PCR technique (multiplex real-time PCR), performed in a single sealed test tube, using TaqMan type molecular probes, specially designed with bioinformatic methods, was developed. The probes employed LNA innovative technology, which had not been used in the tests for the presence of HPV before.

Preferably, the reduction of the length of the probes to 8-10 nucleotides allowed for a significant decrease of their number, from 15-16 to merely several (optimally 2-3). Preferably, it also enabled the carrying out of a multiplex real-time PCR reaction in a single test tube in contrary to most of the tests present on the market, which are carried out in several test tubes. In one embodiment of the invention, LNA probes were optimally labelled with maximally 3 different universal fluorescent dyes with different emission spectra of fluorescent light, which allows for performing the tests in most commercially available PCR apparatuses, and not only in devices designed for the test of a specific producer.

The invention provides an oligonucleotide characterised by a sequence selected from sequences ID no. 1 to 32, defined in the list of sequences presented below.

The invention provides a kit for detecting HPV by the method of a polymerase chain reaction, characterised in that it comprises:
at least one pair of primers selected from the following oligonucleotide sequences:
   a) for HPV 16: sequence ID no. 1 and sequence ID no. 14
   b) for HPV 18: sequence ID no. 2 and sequence ID no. 15
   c) for HPV 33/33n: sequence ID no. 3 and sequence ID no. 14
   d) for HPV 31: sequence ID no. 4 and sequence ID no. 14
   e) for HPV 35: sequence ID no. 5 and sequence ID no. 14
   f) for HPV 39: sequence ID no. 6 and sequence ID no. 14
   g) for HPV 45: sequence ID no. 7 and sequence ID no. 15
   h) for HPV 51: sequence ID no. 8 and sequence ID no. 16
   i) for HPV 52: sequence ID no. 9 and sequence ID no. 14
   j) for HPV 56/66: sequence ID no. 10 and sequence ID no. 14
   k) for HPV 58: sequence ID no. 11 and sequence ID no. 14
   l) for HPV 59: sequence ID no. 12 and sequence ID no. 17
   m) for HPV 68: sequence ID no. 13 and sequence ID no. 18.

In a preferred embodiment, the kit of the invention is characterised in that it comprises all the oligonucleotides defined above.

Preferably, the kit of the invention further comprises an oligonucleotide probe having a sequence selected from the sequences ID no.: 25 to 28.

Preferably, the kit of the invention further comprises an oligonucleotide probe having a sequence selected from the sequences ID no.: 29 to 32.

Preferably, the kit of the invention further comprises a pair of primers, as an internal control, having an oligonucleotide sequence selected from sequences ID no. 23 to 24.

The invention provides a process for detecting HPV, characterised in that a polymerase chain reaction using at least one pair of primers selected from the oligonucleotide sequences as defined above is conducted, wherein the presence of the virus is evidenced by the amplification of a product comprising its specific pair of primers.

In a preferred embodiment of a process according to the invention, preferably a multiplex real-time polymerase chain reaction with the single-stage use of all the oligonucleotides mentioned above is conducted.

In a preferred embodiment of the process according to the invention, preferably an oligonucleotide probe having a sequence selected from sequences ID no. 25 to 28 is further applied.

In a preferred embodiment of the process according to the invention, preferably an oligonucleotide probe having a sequence selected from sequences ID no.: 29 to 32 is further applied.

In a preferred embodiment of the invention, preferably a pair of primers having an oligonucleotide sequence selected from sequences ID no: 23 to 24 is further applied as an internal control.

In order to implement the invention, a kit of primers and probes allowing for identification and amplification of homologous regions in all oncogenic HPV types and, preferably, an internal control, being a selected sequence of human DNA and/or a synthetic fragment of DNA, was designed and fitted. In one embodiment of the invention, preferably, the reaction was conducted in a single test tube, and preferably, the readout of the reaction was performed in maximally 4 detection channels used in the majority of real-time PCR apparatuses present on the market.

Two 10-nucleotide LNA-TaqMan probes allowing for detection of infections with high-risk HPV (HR-HPV) in one real-time PCR reaction were designed. The first of them, HRP (pan-HPV), is complementary to all oncogenic HPV viruses, while the second one, HRP33n, is complementary to the identified variant of HPV33n, comprising polymorphism of one A/G nucleotide at the site where the probe is attached. Preferably, the 5′ ends of HRP pan-HPV and HRP33n probes were labelled with a FAM (fluorescein) fluorescent dye, while the 3′ ends with a BHQI quencher (Black Hole Quencher 1). The readout of a real-time PCR for HRP pan-HPV and HRP33n probes was performed at a wavelength of 530 nm in a channel designed to read out FAM dye.

Moreover, preferably, an internal control for the PCR test was designed. A system consisting of a synthetic short DNA fragment was used and an oligonucleotide comprising 77 base pairs was designed as a target. A system of 4 primers and a 10-nucleotide LNA-TaqMan (ICP) probe was designed for amplification and detection of the synthetic internal control. An optimal combination of 2 primers for the PCR was selected. The ICP probe was labelled with ROX dye at its 5′ end, and with a BHQ2 quencher at its 3′ end. The readout of the real-time PCR for the ICP probe was performed at a wavelength of 610 nm (channel of the ROX dye).

The aim of the study was to possibly design a small kit of primers which would enable the efficient amplification of all oncogenic HPV types in clinical samples with sufficient sensitivity and specificity, with a possible complete elimination of any adverse effects which may occur during PCR, preferably conducted in real time, preferably in several detection channels.

As a result of conducted research, a common pool of primers for high-risk HPV was developed. The sequences were selected in a way so that the total number of primers was as small as possible. Therefore, some sequences of primers are common to several HPV types. Thus, the common reverse primer (“reverse”) for 16, 31, 33, 33n, 35, 39, 52, 56, 58, HPV types and the common reverse primer (“reverse”) for 18 and 45 HPV types were selected. Furthermore, common forward primers (“forward”) were selected for 56 and 66 types and 33 and 33n types, respectively.

According to the embodiment of the invention, preferably, the final mixture of the HR-HPV test of the invention comprises 18 primers (13 forward primers and 5 reverse primers) and 2 LNA-TaqMan probes for the detection of 15 oncogenic HPV types, 2 primers and 1 TaqMan probe for synthetic internal control.

Using selected clinical samples, previously examined with the well-known PapilloCheck test, which detects 24 types of HPV (approx. 50 clinical samples containing the DNA of a single oncogenic HPV type, 30 clinical samples containing the DNA of a non-oncogenic HPV type, 50 clinical samples containing co-infections and 30 negative samples (free from 24 types of HPV, including all oncogenic types), it was demonstrated that an adequate combination of primers and molecular probes, according to the invention, allows for the specific detection of oncogenic HPV types (including the HPV33n variant) and produces negative results in the case of clinical samples containing a non-oncogenic virus type and negative clinical samples.

Moreover, the reaction using the above-mentioned 24 primers, 3 LNA probes and one internal control (IC) TaqMan Probe did not generate products of the type primer-dimer or other non-specific PCR amplification products.

### BRIEF DESCRIPTION OF THE SEVERAL VIEWS OF THE DRAWINGS

An embodiment of the subject of the invention was shown in the drawings.

FIG. 1 is a graph illustration presenting the results obtained in an exemplary test using a real-time PCR for all groups of oncogenic HPVs, with a positive control sample, negative control sample and negative clinical sample. A detection channel of 530 nm was used.

FIG. 2 is a graph illustration showing the results obtained in an exemplary embodiment of a test using a real-time PCR for 16 and 18 HPV types, with a positive control sample, negative control sample and negative clinical sample. A detection channel of 560 nm was used.

FIG. 3 is a graph illustration showing the results obtained in an exemplary embodiment of a test using a real-time PCR for the internal control (IC) of the test, with a positive control sample, negative control sample and negative clinical sample. A detection channel of 610 nm was used.

FIGS. 4A-D are illustrations showing sequences of example probes and primers according to various embodiments of the invention, and corresponding regions of the HPV genome to which they hybridise.

FIG. 4A: shows a region of the HPV16 El gene (SEQ ID NO: 33) that is amplified and targeted by the HR-HPV (pan-HPV) probe (SEQ ID NO: 25) for detection of HPV 16, 18, 31, 33, 35, 39, 45, 51, 52, 56, 66, 58, 59 and 68; the target sequence to which the probe hybridizes (SEQ ID NO: 34) is shown in bold and underlined. The sequence of the HR-HPV (pan-HPV) Probe (5′-actttcgttt-3′, SEQ ID NO: 25/29), which is the reverse complement of the target sequence, is also shown.

FIG. 4B shows a region of the HPV 33n El gene (SEQ ID NO: 34) that is amplified and targeted by the HPV 33n probe for detection of HPV33n; the target sequence (SEQ ID NO: 36) to which the probe hybridizes is shown in bold and underlined). The sequence of the HR-HPV 33n Probe (5′-actttcgctt-3′, SEQ ID NO: 26/30) which is the reverse complement of the target sequence, is also shown.

FIG. 4C shows the regions of the HPV 16 El gene (SEQ ID NO: 37) and HPV 18 El gene (SEQ ID NO: 40) that are amplified for targeting by the HPV16/18 probe. The target sequence to which Primer HPV 16 F HEX (SEQ ID NO: 19) and Primer HPV 16 R HEX (SEQ ID NO: 20) hybridise for amplification and the target sequence (SEQ ID NO: 71) to which the HPV 16/18 probe (SEQ ID NO: 38) hybridizes are shown in bold and underlined. The target sequences to which Primer HPV 18 F HEX (SEQ ID NO: 21) and Primer HP VI 8 HEX (SEQ ID NO: 22) hybridise for amplification and the target sequence to which the HPV16/18 probe (SEQ ID NO: 39) hybridizes are shown in bold and underlined. Note that the HPV16/18 probe shown (5′-atatctgatgacgag-3′ (SEQ ID NO: 39)) has a “t” nucleotide as marked, whereas the HPV16/18 probe SEQ ID NO: 38 has an “a” residue at this position. The reverse primer HPV 18 R HEX has an “a” nucleotide where indicated, whereas for the target sequence shown, the complement would normally be a “g” nucleotide; the native HPV sequence has a “t” nucleotide at this position, hence the reverse primer used to target this sequence has an “a” at this position.

FIG. 4D also shows the region of the HPV16 El gene (SEQ ID NO: 41) that is amplified and targeted by the HR-HPV (pan-HPV) probe for detection of HPV 16, 18, 31, 33, 35, 39, 45, 51, 52, 56, 66, 58, 59 and 68. The target sequence to which the primers HPV 16 F (SEQ ID NO: 1) and HPV 16/31/33/33n/35/39/55/56/58/66 R (SEQ ID NO: 14) hybridise are shown in bold and underlined. The target sequence (SEQ ID NO: 34) to which the pan-HPV probe (SEQ ID NO: 25) hybridizes is shown in bold and underlined.

FIGS. 5A-N are illustrations showing sequences of the high risk HPV genome El regions for HPV strains 16, 18, 33, 31, 35, 39, 45, 51, 52, 56, 58, 59, 66 and 68, with the sequences of probes and primers according to various embodiments of the invention, and corresponding regions of the HPV genome to which they hybridise for detection in the first detection channel 530 nm FAM. FIG. 5A shows the HPV 16 El gene nucleotides 865 to 2814 (SEQ ID NO: 44), marked to show the positions of the Forward primer HPV 16 (SEQ ID NO: 1) 5′-gcacaggaagcaaaacaacatag-3′ and the region of the HPV 16 El gene targeted by the reverse primer HPV 16/31/33/33n/35/39/52/56/58/66R 5′-agacagcgggtatggcaatac-3′ (SEQ ID NO: 45). Also shown is the Reverse primer HPV16/31/33/33n/35/39/52/56/58/66R 5′-gtattgccatacccgctgtct-3′ (SEQ ID NO: 14) which is the reverse complement of the region of the HPV 16 El gene shown in SEQ ID NO: 45. The 5′-aaacgaaagt-3′ sequence is the pan-HPV Probe target sequence (SEQ ID NO: 34) for hybridization of the 5′-actttcgttt-3′ pan-HPV Probe (SEQ ID NO: 25, which is the reverse complement of SEQ ID NO: 34). The target region of HPV16 amplified for detection with the pan-HPV probe is nucleotides 1081 to 1274.

FIG. 5B shows the HPV 18 El gene nucleotides 914 to 2887 sequence (SEQ ID NO: 46), marked to show the positions of the Forward primer HPV 18 5′-aggtccacaatgatgcacaag-3′ (SEQ ID NO: 2) and the target region of the HPV18 El genome 5′-tcagatagtggctatggctgtt-3′ (SEQ ID NO: 47) that is targeted by the reverse primer. The Reverse primer HPV 18/45 R 5′-aacagccatagccactatctga-3′ (SEQ ID NO: 15), is shown, this is the reverse complement of the target region of the HPV18 El genome shown in SEQ ID NO: 47. Also shown is the 5′-aaacgaaagt-3′ (SEQ ID NO: 34) pan-HPV probe target sequence for hybridization of the 5′-actttcgttt-3′ (SEQ ID NO: 25) pan-HPV Probe (SEQ ID NO: 34 is the reverse complement of SEQ ID NO: 25). The target region of HPV16 amplified for detection using the pan-HPV probe is nucleotides 1134 to 1331.

FIG. 5C shows the HPV 33 El gene nucleotides 879 to 2813 (SEQ ID NO: 48) marked to show the Forward primer for HPV 33 5′-cagaagatgaggatgaaacagcag-3′ (SEQ ID NO: 3), and the target binding site of the reverse primer HPV16/31/33/33n/35/39/52/56/58/66R 5′agacagcggatatggcaatac3′(SEQ ID NO: 49) in which there is a base difference a/g as marked relative to the target sequence for the reverse primer HPV 16/31/35/39/52/56/58/66R, thus 5′-gtattgccatatccgctgtct-3′ (SEQ ID NO: 50) shows a mismatch c/t in the reverse complement when compared to the primer (SEQ ID NO: 14) that is used for amplification. The sequence 5′-aagcgaaagt-3′ (SEQ ID NO: 36) is the HR-HPV 33n region targeted by the HPV 33n Probe, this has a nucleotide difference a/g compared to the consensus region targeted by the pan-HPV probe (SEQ ID NO: 25). The sequence 5′-actttcgctt-3′ (SEQ ID NO: 26) is the HR-HPV Probe 33n (reverse complement of SEQ ID NO: 36) which has a nucleotide difference t/c when compared to the pan-HPV probe (SEQ ID NO: 25). The sequence 5′-aaacgaaagt-3′ (SEQ ID NO: 34) is the target sequence for the pan-HPV Probe used for detection of HPV 33. The sequence 5′-actttcgttt-3′ (SEQ ID NO: 25) is the pan-HPV Probe used for detection of HPV 33 (SEQ ID NO: 25 is the reverse complement of SEQ ID NO: 34). The target region of HPV 33 (and 33n) amplified for detection using the pan-HPV probe is nucleotides 976 to 1285.

FIG. 5D shows the HPV 31 El gene nucleotides 862 to 2751 nucleotides (SEQ ID NO: 51), marked to show the positions of the Forward primer HPV 31 5′-gaggaacatgcagaggctgt-3′ (SEQ ID NO: 4) and the target sequence for the Reverse primer HPV16/31/33/33n/35/39/52/56/58/66R 5′-agacagcgggtatggcaatac-3′ (SEQ ID NO: 45). The sequence 5′-gtattgccatacccgctgtct-3′ (SEQ ID NO: 14) is the reverse primer for HPV16/31/33/33n/35/39/52/56/58/66R (SEQ ID NO: 14 is the reverse complement of the target region shown in SEQ ID NO: 45). The sequence 5′-aaacgaaagt-3′ (SEQ ID NO: 34) is the target sequence for the pan-HPV Probe used for detection of HPV 31. The sequence 5-actttcgttt-3′ (SEQ ID NO: 25) is the pan-HPV Probe used for detection of HPV 31 (SEQ ID NO: 25 is the reverse complement of SEQ ID NO: 34). The target region amplified for detection of HPV 31 is nucleotides 1087 to 1268.

FIG. 5E shows the HPV 35 El gene nucleotides 868 to 2781 nucleotides (SEQ ID NO: 52), marked to indicate the positions of the Forward primer HPV 35 5′-atgcacaggaggagcaaaca-3′ (SEQ ID NO: 5), and the target region for the Reverse primer HPV16/31/33/33n/35/39/52/56/58/66R, the genomic sequence 5′-agacagcggttatggcaattc-3′ (SEQ ID NO: 53) shows the two nucleotide differences t/g and t/a between the target sequence of HPV35 and the target sequence for the reverse primer. The sequence 5′-gaattgccataaccgctgtca-3′ (SEQ ID NO: 54), complementary to SEQ ID NO: 53, shows the two nucleotide differences t/a and a/c relative to the reverse primer (SEQ ID NO: 14). The sequence 5′-agacagcgggtatggcaatac-3′ (SEQ ID NO: 45) is the target sequence for the reverse primer HPV16/31/33/33n/35/39/52/56/58/66R. The sequence 5′-gtattgccatacccgctgtct-3′ (SEQ ID NO: 14) is the Reverse primer HPV16/31/33/33n/35/39/52/56/58/66R used for amplification of HPV 35 (SEQ ID NO: 14 is the reverse complement of SEQ ID NO: 45). The sequence 5′-aaacgaaagt-3′ (SEQ ID NO: 34) is the target consensus sequence for the pan-HPV Probe used for detection of HPV 35. The sequence 5′-actttcgttt-3′ (SEQ ID NO: 25) is the pan-HPV Probe used for detection of HPV 35 (which is the reverse complement of SEQ ID NO: 34). The target region of the HPV 35 El gene amplified for detection is nucleotides 1082 to 1276.

FIG. 5F shows the HPV39 El gene nucleotides 928 to 2871 (SEQ ID NO: 55), marked to show the position of the Forward primer 39F 5′-gtgagacagcacaggtacttttac-3′ (SEQ ID NO: 6) and the target region for the Reverse primer HPV16/31/33/35/39/52/56/58/66R 5′-agacagcggatatggcaatac-3′ (SEQ ID NO: 49) which shows the nucleotide difference a/g between the target sequence of HPV 39 and the target sequence for the reverse primer. The sequence 5′-gtattgccatatccgctgtct-3′ (SEQ ID NO: 50) shows the nucleotide difference c/t in the reverse complement of SEQ ID NO: 49, the reverse primer used for amplification of HPV16/31/33/35/39/52/56/58/66R (SEQ ID NO: 14) has a “c” nucleotide at the marked position. The sequence 5′-aaacgaaagt-3′ (SEQ ID NO: 34) is the target consensus sequence for the pan-HPV Probe used for detection of HPV 39. The sequence 5′-actttcgttt-3′ (SEQ ID NO: 25) is the pan-HPV Probe used for detection of HPV 39 (SEQ ID NO: 25 is the reverse complement of SEQ ID NO: 34). The target amplified for detection of HPV 39 is nucleotides 1115-1339.

FIG. 5G shows the HPV 45 El gene nucleotides 914 to 2845 (SEQ ID NO: 56), marked to indicate the Forward primer HPV 45 F 5′-ccatgcgcaggaagttcag-3′ (SEQ ID NO: 7), and the target region for the Reverse primer HPV 18/45 R 5′-tcagatagtggctatggctgtt-3′ (SEQ ID NO: 47), this region is targeted by the reverse primer for both HPV 18 and 45. The sequence 5′-aacagccaatagccactatctga-3′(SEQ ID NO: 15) is the reverse primer for HPV 18/45 R (reverse complement of SEQ ID NO: 47). The sequence 5′-aaacgaaagt-3′ is the target consensus sequence for the pan-HPV Probe used for detection of HPV 45. The sequence 5′-actttcgttt-3′ (SEQ ID NO: 25) is the pan-HPV Probe used for detection of HPV 45 (SEQ ID NO: 25 is the reverse complement of SEQ ID NO: 34). The target amplified for detection of for HPV 45 is nucleotides 1123-1331.

FIG. 5H shows the region of the HPV51 genome that contains the HPV51 gene (SEQ ID NO: 57), marked to show the Forward primer HPV 51 F 5′-acaaagaggctgtgcatcag-3′ (SEQ ID NO: 8) and the target sequence for the Reverse primer HPV 51 R 5′-actggacagttatccggacag-3′ (SEQ ID NO: 58). The sequence 5′-ctgtccggataactgtccagt-3′ (SEQ ID NO: 16) is the reverse primer for HPV 51 R (reverse complement of SEQ ID NO: 58). The sequence 5′-aaacgaaagt-3′ (SEQ ID NO: 34) is the target consensus sequence for the pan-HPV Probe used for detection of HPV 51. The sequence 5′-actttcgttt-3′ (SEQ ID NO: 25) is the pan-HPV Probe used for detection of HPV 51 (SEQ ID NO: 25 is the reverse complement of SEQ ID NO: 34). The target HPV 51 El region amplified for detection is nucleotides 1103-1256.

FIG. 5I shows the HPV 52 El gene nucleotides 864 to 2807 marked to show the positions of the Forward primer HPV 52 5′-gaaggggaggatgatttacatgc-3′ (SEQ ID NO: 9) and the target region for the Reverse primer HPV16/31/33/33n/35/39/52/56/58/66R 5′-agacagcggctatggcaatag-3′(SEQ ID NO: 60), there are two nucleotide differences g/c and c/g as shown between the HPV52 target sequence and the target sequence for the reverse primer. The sequence 5′ctattgccatagccgctgtct-3′ (SEQ ID NO: 61) is the reverse complement of SEQ ID NO: 60, there are two nucleotide differences as shown, c/g and g/c, between the reverse complement and the Reverse primer HPV16/31/33/33n/35/39/52/56/58/66R (SEQ ID NO: 14) used for amplification of HPV52. The sequence 5′-aaacgaaagt-3′ (SEQ ID NO: 34) is the target consensus sequence for the pan-HPV Probe used for detection of HPV 52. The sequence 5′-actttcgttt-3′ (SEQ ID NO: 25) is the pan-HPV Probe used for detection of HPV 52 (SEQ ID NO: 25 is the reverse complement of SEQ ID NO: 34). The target region amplified for detection of the HPV 52 El gene is nucleotides 1083-1269.

FIG. 5 J shows the HPV 56 El gene nucleotides 895 to 2805 (SEQ ID NO: 62), marked to show the positions of the Forward primer HPV 56/66 5′-gcaagtacaaacagcacatgc-3′ (SEQ ID NO: 10) and the target for the Reverse primer HPV 16/31/33/33n/35/39/52/56/58/66R 5′-agacagcgggtatggcaatac-3′ (SEQ ID NO: 45). The sequence 5′-gtattgccatacccgctgtct-3′ (SEQ ID NO: 14) is the sequence of the reverse primer for HPV 16/31/33/33n/35/39/52/56/58/66R (reverse complement of SEQ ID NO: 45). The sequence 5′-aaacgaaagt-3′ (SEQ ID NO: 34) is the target consensus sequence for the pan-HPV Probe used for detection of HPV 56. The sequence 5′-actttcgttt-3′ (SEQ ID NO: 25) is the pan-HPV Probe used for detection of HPV 52 (the reverse complement of SEQ ID NO: 34). The target amplified for detection of HPV 56/66 is nucleotides 1104 to 1267.

FIG. 5K shows the HPV66 El gene nucleotides 895 to 2787 nucleotides (SEQ ID NO: 63), marked to indicate the position of the Forward primer HPV 56/66 5′-gcaagtacaaacagcacatgc-3′ (SEQ ID NO: 10) and the target for the Reverse primer HPV16/31/33/33n/35/39/52/56/58/66R 5′-agacagigggtatggcaatac-3′ (SEQ ID NO: 64), note the nucleotide difference c/t between the target sequence for the reverse primer and the target sequence of HPV 66. The sequence 5′-gtattgccataccc ctgtct-3′ (SEQ ID NO: 65) shows the nucleotide difference g/a between the reverse complement of SEQ ID NO: 64 and the reverse primer HPV16/31/33/33n/35/39/52/56/58/66R (SEQ ID NO: 14) that is used for amplification of HPV 66. The sequence 5′-aaacgaaagt-3′ (SEQ ID NO: 34) is the target consensus sequence for the pan-HPV Probe used for detection of HPV 66. The sequence 5′-actttcgttt-3′ (SEQ ID NO: 25) is the pan-HPV Probe used for detection of HPV 56 (the reverse complement of SEQ ID NO: 34). The target region of the HPV 56/66 El gene is nucleotides 1104-1262.

FIG. 5L shows the HPV 58 El gene nucleotides 883 to 2817 (SEQ ID NO: 66), marked to show the positions of the Forward primer 58 F 5′-agcccgagcgttgtttaatg-3′ (SEQ ID NO: 11), and the target region for the Reverse primer HPV16/31/33/33n/35/39/52/56/58/66R. The sequence 5′-agacagcggatatggcaatac-3′ (SEQ ID NO: 49), shows the nucleotide difference g/a relative to the target sequence for the reverse primer for HPV16/31/33/33n/35/39/52/56/58/66R. The sequence 5′-gtattgccatafccgctgtct-3′ (SEQ ID NO: 50) shows the nucleotide difference c/t between the reverse complement of SEQ ID NO: 49 and the reverse primer for HPV16/31/33/33n/35/39/52/56/58/66R that is used to amplify HPV 58. The sequence 5′-aaacgaaagt-3′ (SEQ ID NO: 34) is the target consensus sequence for the pan-HPV Probe used for detection of HPV 58. The sequence 5′-actttcgttt-3′ (SEQ ID NO: 25) is the pan-HPV Probe used for detection of HPV 58 (the reverse complement of SEQ ID NO: 34). The target amplified for detection of HPV 58 is nucleotides 1077 to 1289.

FIG. 5M shows the HPV 59 El gene nucleotides 872 to 2806 (SEQ ID NO: 67) marked to show the position of the Forward primer HPV 59 F 5′-ggccttgtttaatgtgcaggaag-3′ (SEQ ID NO: 12) and the region targeted by the Reverse primer HPV 59 R 5′-gaaggttamtaacagtgccagaca-3′ (SEQ ID NO: 68), in which m denotes a nucleotide change c/a. The sequence 5′-tgtctggcactgttaktaaccttc-3′ (SEQ ID NO: 17) is the reverse primer used for amplification of HPV 59 (reverse complement of SEQ ID NO: 68), k denotes a change of nucleotide g/t. The sequence 5′-aaacgaaagt-3′ (SEQ ID NO: 34) is the target consensus sequence for the pan-HPV Probe used for detection of HPV 59. The sequence 5′-actttcgttt-3′ (SEQ ID NO: 25) is the pan-HPV Probe used for detection of HPV 59 (the reverse complement of SEQ ID NO: 34). The target region amplified for detection of the HPV 59 El gene is nucleotides 1172-1265.

FIG. 5N shows HPV 68 El nucleotides 824 to 2746 (SEQ ID NO: 69) marked to show the position of the Forward primer HPV 68 F 5′-caaacaggtgacacagtctcag-3′ (SEQ ID NO: 13) and the target sequence for the Reverse primer HPV 68 R 5′-agcaaagtcgccattacagga-3′ (SEQ ID NO: 70). The sequence 5′-tcctgtaatggcgactttgct-3′ (SEQ ID NO: 18) is the reverse primer for HPV68 (the reverse complement of SEQ ID NO; 70). The sequence 5′-aaacgaaagt-3′ (SEQ ID NO: 34) is the target consensus sequence for the pan-HPV Probe used for detection of HPV 68. The sequence 5′-actttcgttt-3′ (SEQ ID NO: 25) is the pan-HPV Probe used for detection of HPV 68 (the reverse complement of SEQ ID NO: 34). The target region amplified for detection of HPV 68 El is nucleotides 902-1137.

FIGS. 6A and 6B are illustrations showing the sequences of the high risk HPV genome El regions for HPV strains 16 and 18 with the sequences of probes and primers according to various embodiments of the invention, and corresponding regions of the HPV genome to which they hybridise for detection in the second detection channel 560 nm HEX.

FIG. 6 A shows the HPV 16 El gene sequence nucleotides 865 to 2814 nucleotides (SEQ ID NO: 44) marked to show the position of the Forward primer HPV 16 F HEX 5′taatggatggttttatgtagaggct-3′ (SEQ ID NO: 19) for amplification of HPV16 for the second detection channel (560 nm) and the target sequence for the Reverse primer HPV 16 R HEX 5-ataatgattatttaacacaggcag-3′ (SEQ ID NO: 71). The sequence 5′-ctgcctgtgttaaataatcattat-3′ (SEQ ID NO: 20) is the HPV 16 R HEX primer sequence (SEQ ID NO: 20 is the reverse complement of SEQ ID NO: 71). The sequence 5′-atatcagatgacgag-3′ (SEQ ID NO: 27/31/38) is sequence of the HR HPV 16/18 Probe. The target for amplification and detection of HPV 16 in the second detection channel (560 nm HEX) is nucleotides 909-1051.

FIG. 6B shows the HPV 18 El gene nucleotides 914 to 2887 (SEQ ID NO: 46) marked to show the positions of the Forward primer HPV 18 F HEX 5′-ggctggttttatgtacaagct-3′ (SEQ ID NO: 21) and the region targeted by the Reverse primer HPV 18 R HEX 5′-aaggaacattttgfgaacagg-3′ (SEQ ID NO: 72), note there is a nucleotide difference c/t relative to the corresponding HPV 18 El genomic sequence shown in SEQ ID NO: 46. The sequence 5′-cctgttcacaaaatgttcctt-3′ reverse complement (SEQ ID NO: 22) is the HPV18R HEX primer sequence use for amplification of HPV 18 in the second detection channel (560 nm HEX). The sequence 5′-atatc¾atgacgag-3′ (SEQ ID NO: 27/31/39) is the sequence of the HPV 18 showing the nucleotide difference A/T relative to the HR HPV 16/18 Probe 5′-atatcagatgacgag-3′ (SEQ ID NO: 27/31/38). The target region for HPV 18 in the second detection channel (560 nm HEX) is nucleotides 959-1097.

### DETAILED DESCRIPTION OF THE INVENTION

As described above, and presented in more detail in the accompanying examples and drawings, one aspect of the invention is a composition comprising a nucleic acid probe for HPV disclosed herein. The probe may be labelled with any label suitable for use in real time PCR. Typical labels are a fluorophore and quencher, located at opposite ends of the linear probe, for example the fluorophore may be attached at the 5′ end of the probe and the quencher at the 3′ end. The probe may have a nucleotide sequence comprising or consisting of a probe sequence set out herein, or its reverse complement sequence (which corresponds to the target sequence of the El region of the HPV genome to which the probe binds). For example, the nucleotide sequence may be the pan-HP V probe ACTTTCGTTT (SEQ ID NO: 25) which is also referred to herein as the HR-HPV probe. The reverse complement of this sequence is AAACGAAAGT (SEQ ID NO: 42).

Preferably the probe is an LNA probe molecule, in which the nucleotide sequence comprises LNA nucleotides. Advantageously, probes comprising LNA allow a shorter sequence length to be employed while still conferring high specificity of hybridisation. HPV probes according to the present invention are generally 8, 9, 10, 11, 12, 13, 14, 15 or 16 nucleotides in length. For example, probes may be up to 15 nucleotides in length and comprise a probe sequence set out herein or its reverse complement. As noted above, LNA nucleotides contain an additional methylene bridge within the molecule of deoxyribose. These additional methylene bridges are optionally present in at least 8, 9 or 10 of the nucleotides in the probe. Methylene bridges may be absent in some nucleotides in the probe. Examples of exact locations of methylene bridges in probe sequences are presented in Example 1 (d) below.

One of the key findings underlying the present invention is that such short LNA probes may be directed to regions of homology among oncogenic HPV types, and may hybridise with sufficient specificity that they permit a large range of oncogenic HPV strains to be specifically detected using a single probe or a small number of probes such as 1, 2 or 3 probes. Thus, a probe according to the present invention may be a pan-HPV probe, able to hybridise to a plurality of oncogenic HPV. Oncogenic HPV strains include HPV16, HPV18, HPV31, HPV33, HPV33n, HPV35, HPV39, HPV45, HPV51, HPV52, HPV56, HPV58, HPV59, HPV66 or HPV68. Preferably, pan-HPV probes according to the present invention target a region of the HPV genome in the El gene, for example as shown below in Table 1.

TABLE 1

| | Target region of the HPV E1 gene for |
|---|---|
| | amplification/detection (e.g., in first detection |
| HPV strain | channel 530 nm FAM) |
| 16 | 1081-1274 |
| 18 | 1134-1331 |
| 31 | 1087-1268 |
| 33/33n | 976-1285 |
| 35 | 1082-1276 |
| 39 | 1115-1339 |
| 45 | 1123-1331 |
| 51 | 1103-1256 |
| 52 | 1083-1269 |
| 56 | 1104-1267 |
| 58 | 1077-1289 |
| 59 | 1172-1265 |
| 66 | 1104-1262 |
| 68 | 902-1137 |

Preferably, HPV 16/18 probes according to the present invention target a region of the HPV genome in the El gene, for example as shown below in Table 2.

TABLE 2

| | Target region of the HPV E1 gene for |
|---|---|
| | amplification/detection (e.g., in second |
| HPV strain | detection channel 560 nm HEX) |
| 16 | 909-1051 |
| 18 | 959-1097 |

The probes described herein may be provided for use in detecting HPV, including as part of diagnostic methods to detect the presence of oncogenic HPV in samples or to specifically determine whether a sample tests positive or negative for oncogenic HPV. A sample may be any material to be tested for oncogenic HPV, such as a sample comprising biological material from a subject e.g., from a human patient. The biological material may be obtained from a cervical smear. Preferably, the sample is a liquid-based cytology sample from a human cervical smear. The probes are optimised for use in HPV detection by real-time PCR. Primers suitable for use in combination with the probes are also described and represent further aspects of the invention.

Accordingly, one aspect of the invention is a method of determining whether a sample tests positive for oncogenic HPV, the method comprising:
combining the sample with primers for real time PCR amplification of a target region of oncogenic HPV genomes, and a labelled pan-HPV probe capable of emitting a signal upon amplification of the target region,
providing conditions for real time PCR, and
measuring for a signal emitted from the labelled pan-HPV probe,
wherein detection of the signal indicates that the sample tests positive for oncogenic HPV.

Examples of pan-HPV probes are described herein, such as the labelled LNA molecule comprising sequence ACTTTCGTTT (SEQ ID NO: 25) or its reverse complement. The pan-HPV probe may be a labelled 10-nucleotide molecule of sequence consisting of ACTTTCGTTT (SEQ ID NO: 25).

Examples of suitable primers are also described herein. Preferably the primers are for amplification of a target region of any of HPV16, HPV18, HPV31, HPV33, HPV35, HPV39, HPV45, HPV51, HPV52, HPV56, HPV58, HPV59, HPV66 or HPV68. The method may comprise combining the sample with a combination of primers for amplification of all of these HPV types, thus allowing detection of any such HPV if it is present in the sample.

As the pan-HPV probe may be one that is designed to specifically detect any HPV belonging to a particular set of oncogenic HPV (e.g., the set of HPV16, HPV18, HPV31, HPV33, HPV35, HPV39, HPV45, HPV51, HPV52, HPV56, HPV58, HPV59, HPV66 and HPV68), detection of a signal from the labelled pan-HPV probe in an assay with all such primers therefore indicates that the sample tests positive for at least one of these oncogenic HPV, whereas the absence of a signal in these circumstances indicates that the sample tests negative for these oncogenic HPV. The test may not provide absolute certainty, in view of the limits of detection of the assay, discussed in the examples below, but methods of the invention have been shown to provide excellent accuracy and consistency with alternative methods of testing for oncogenic HPV. Methods of the invention may, if desired, be combined with one or more further techniques, such as visual examination of cells in the sample, to provide additional confidence as to the presence or absence of oncogenic HPV.

Use of a plurality of probes of different HPV specificity can extend the range of HPV detected and/or provide information regarding the type of HPV present in the sample.

For example, the pan-HPV probe of SEQ ID NO: 25 may bind more weakly to the target region of HPV33n than the target region of other oncogenic HPV, due to differences in the HPV33n genome sequence in this region. Accordingly, the invention provides a supplementary probe for HPV 33n to hybridise more strongly to the target region in HPV33n. Exemplary 33n probes described herein comprise sequence ACTTTCGCTT (SEQ ID NO: 26) or its reverse complement. The 33n probe may be a labelled 10-nucleotide molecule of sequence consisting of ACTTTCGCTT (SEQ ID NO: 26). It may be an LNA probe. A probe for HPV33n may be combined with a pan-HPV probe in methods of the invention. Conveniently, if both probes are identically labelled (e.g., with the same fluorophore) then they will emit a common signal (e.g., fluorescence of the same wavelength). Detection of this signal then indicates the presence of any HPV bound by the pan-HPV probe or the 33n probe, for instance it may indicate the presence of any oncogenic HPV belonging to the set of HPV16, HPV18, HPV31, HPV33, HPV33n, HPV35, HPV39, HPV45, HPV51, HPV52, HPV56, HPV58, HPV59, HPV66 and HPV68.

Thus, a method of determining whether a sample tests positive for oncogenic HPV may comprise
additionally combining the sample with primers for amplification of a target region of HPV33n, and a labelled 33n probe capable of emitting a signal upon amplification of the target region, wherein the 33n probe and the pan-HPV probe carry the same label,
providing conditions for-real time PCR, and
measuring for a signal emitted from the labelled 33n probe or the pan-HPV probe, wherein detection of the signal indicates that the sample tests positive for oncogenic HPV.

Another aspect of the invention is a method of specifically testing a sample for HPV16 and/or HPV18. Detection of these strains of HPV is of particular importance, as it is believed that HPV 16 and HPV 18 have the highest oncogenic potential and are associated with the greatest risk of cervical cancer. A method of detecting HPV16/18 may be performed alone, but it is advantageous to combine it with the pan-HPV detection method above, to provide the additional information as to whether HPV present in the sample is of the type HPV 16 or HPV 18 or belongs to a different strain of the virus.

Accordingly the method of determining whether a sample tests positive for oncogenic HPV may comprise additionally determining whether the sample tests positive for HPV16 or HPV18, and may comprise:
combining the sample with primers for real time PCR amplification of a target region of HPV 16 and HPV 18 genomes, and a labelled 16/18 probe capable of emitting a signal upon amplification of the target region,
providing conditions for real time PCR, and
measuring for a signal from the labelled 16/18 probe,
wherein detection of a signal indicates that the sample tests positive for HPV16 or HPV18.

Examples of probes for HPV16 and HPV18 are set out herein, and include an LNA molecule comprising sequence ATATCWGATGACGAG (SEQ ID NO: 27) or its reverse complement.

To facilitate resolution of the HPV 16/18 signal from the pan-HPV signal in a combined detection method, the 16/18 probe may be labelled differently (e.g., with a different fluorophore) so that it emits a different signal (e.g. fluorescence at a different wavelength) from the pan-HPV probe.

Positive and/or negative controls may be included in the method as desired. Typically, conducting a positive control for the real time PCR will comprise
combining the sample with primers for real time PCR amplification of a control DNA sequence (e.g., human DNA) present in the sample, and a labelled control probe capable of emitting a signal upon amplification of the control DNA sequence,
providing conditions for real time PCR, and
measuring for a signal from the control probe,
wherein detection of the signal indicates that the real time PCR is functional.

Preferably, as illustrated in the Examples, methods of the invention are performed in multiplex. Thus, multiple probes (any two or more, or all, of: pan-HPV probe, 33n probe, 16/18 probe and the control probe) and their corresponding primers for the real time PCR may be included in a single reaction mixture. The use of different labels and detection of signals with different channels (e.g., fluorescence at different wavelengths) allows the signals from different probes to be distinguished. The reaction mixture will contain suitable buffers and other reagents for the real-time PCR. Examples of reagents and reaction conditions for PCR are well-known and are provided in the Examples

Embodiments of the invention are presented below.

### Example I

### Embodiment of the Diagnostic Test

In this embodiment, the presence of all studied oncogenic HPV viruses and the presence of 16 and 18 HPV types were determined in a test sample.

Preferably, the positive control sample was prepared of a HPV 16 nucleotide sequence and a fragment of a sequence of human beta globin.

Preferably, one control positive sample and one control negative sample were applied for each assay. In this embodiment, the positive control sample indicates the correctness of the conducted reaction (lack of degradation of the positive control sample, appropriate activity of reagents, correct thermal profile of the device, no inhibition of the reaction). A negative control sample allows for verification of whether a contamination of the DNA eluate and reagents with biological material containing HPV or amplified PCR products occurred during the isolation of DNA and preparation of the amplification reaction. If a positive signal in at least one detection channel is detected for the negative control sample, it means that a contamination of the negative control sample or reagents occurred.

Furthermore, an internal control (IC), comprising a fragment of a sequence of human beta globin applied to confirm whether each clinical test sample contains a sufficient amount of DNA, needed to detect HR HPV, was used. Moreover, a signal from the internal control indicates properly conducted DNA isolation and lack of reaction inhibitors. Preferably, the positive control sample comprises a sequence of the internal control. The negative control sample should produce negative results in three detection channels.

1. Sequences of used primers, probes and control samples of the invention

a) oligonucleotide sequence of the primer for the first FAM channel at 530 nm:

| SEQ ID | | | |
|---|---|---|---|
| NO.: | PRIMER | ORIENTATION | PRIMER SEQUENCE |
| 1 | HPV 16 F | Forward | 5'-GCACAGGAAGCAAAACAACATAG-3' |
| 2 | HPV 18 F | Forward | 5'-AGGTCCACAATGATGCACAAG-3' |
| 3 | HPV 33/33n F | Forward | 5'-CAGAAGATGAGGATGAAACAGCAG-3' |
| 4 | HPV 31 F | Forward | 5'-GAGGAACATGCAGAGGCTGT-3' |
| 5 | HPV 35 F | Forward | 5'-ATGCACAGGAGGAGCAAACA-3' |
| 6 | HPV 39 F | Forward | 5'-GTGAGACAGCACAGGTACTTTTAC-3' |
| 7 | HPV 45 F | Forward | 5'-CCATGCGCAGGAAGTTCAG-3' |
| 8 | HPV 51 F | Forward | 5'-ACAAAGAGGCTGTGCATCAG-3' |
| 9 | HPV 52 F | Forward | 5'-GAAGGGGAGGATGATTTACATGC-3' |
| 10 | HPV 56/66 F | Forward | 5'-GCAAGTACAAACAGCACATGC-3' |
| 11 | HPV 58 F | Forward | 5'-AGCCCGAGCGTTGTTTAATG-3' |
| 12 | HPV 59 F | Forward | 5'-GGCCTT GTTTAATGTGCAGGAAG-3' |
| 13 | HPV 68 F | Forward | 5'-CAACAGGTGACACAGTCTCAG-3' |
| 14 | HPV | Reverse | 5'-GTATTGCCATACCCGCTGTCT-3' |
| | 16/31/33/ | | |
| | 33n/35/39/52/ | | |
| | 56/58/66R | | |
| 15 | HPV 18/45 R | Reverse | 5'-AACAGCCATAGCCACTATCTGA-3' |
| 16 | HPV 51 R | Reverse | 5'-CTGTCCGGATAAGTCTCCAGT-3' |
| 17 | HPV 59 R | Reverse | 5'-TGTCTGGCACTGTTAKTAACCTTC-3' |
| 18 | HPV 68 R | Reverse | 5'-TCCTGTAATGGCGACTTTGCT-3' |

wherein “K” signifies a change of base G/T

b) oligonucleotide sequence of the primer for the second HEX channel at 560 nm:

| SEQ ID | | | |
|---|---|---|---|
| NO.: | PRIMER | ORIENTATION | PRIMER SEQUENCE |
| 19 | HPV 16 F HEX | Forward | 5'-TAATGGATGGTTTTATGTAGAGGCT-3' |
| 20 | HPV 18 R HEX | Reverse | 5'-CTGCCTGTGTTAAATAATCATTAT-3' |
| 21 | HPV 18 F HEX | Forward | 5'-GGCTGGTTTTATGTACAAGCT-3' |
| 22 | HPV 18 R HEX | Reverse | 5'-CCTGTTCACAAAATGTTCCTT-3' |

c) oligonucleotide sequence of the primer for the third ROX channel at 610 nm-internal control:

| SEQ ID | | | |
|---|---|---|---|
| NO.: | PRIMER | ORIENTATION | PRIMER SEQUENCE |
| 23 | HPV IC F | Forward | 5'-GAGAACTTCAAGGTGAGTTCAGGT-3' |
| 24 | HPV IC R | Reverse | 5'-GAAGTCTGCTGTTCTAACATCAAG-3 |

d) oligonucleotide sequence of the probe:

| Probe | Sequence of the probe |
|---|---|
| HR-HPV | 5'-6FAM-A + C + T + T + T + C + |
| (pan-HPV) | G + T + TT-BHQ-1-3' |
| SEQ ID NO.: 29 | |
| 33n | 5'-6FAM-A + C + T + T + T + C + |
| SEQ ID NO.: 30 | G + C + TT-BHQ-1-3' |
| 16/18 | 5'-HEX-AT + AT + CW + GA + T + G + |
| SEQ ID NO.: 31 | AC + G + AG-BHQ-1-3' |
| IC | 5'-ROX-TGACATTAATTGAAGCTCATAATCTTA |
| SEQ ID NO.: 32 | TTG-BHQ-2-3' |

wherein “+” signifies a LNA nucleotide containing an additional methylene bridge

within the deoxyribose; “W” signifies a change of base A/T.

2. Applied concentrations of primers and probes in the reaction mixture of the invention

a) Final concentrations of the primers in the reaction mixture for the first FAM channel at 530 nm equalled:

o. PRIMER Final Initial

concentration in concentration the mixture [μM][μM]

HPV 16 F 0.224

HPV 18 F 0.224

HPV 31 F 0.22 4

HPV 33/33n F 0.22 4

HPV 35 F 0.22 4

HPV 39 F 0.22 4

HPV 45 F 0.22 4

HPV 51 F 0.22 4

HPV 52 F 0.22 4

0 HPV 56/66 F 0.22 4

1 HPV 58 F 0.22 4

2 HPV 59 F 0.22 4

3 HPV 68 F 0.22 4

4 HPV 0.22 4

16/31/33/33n/35/3

9/52/56/58/66R

5 HPV 18/45 R 0.224

6 HPV 51 R 0.22 4

7 HPV 59 R 0.22 4

8 HPV 68 R 0.22 4

b) Final concentrations of the primers in the reaction mixture for the second HEX channel of HPV 16/18 at 560 nm equalled:

| | | Final | Initial |
|---|---|---|---|
| | | concentration in | concentration |
| No. | PRIMER | the mixture [μM] | [μM] |
| 19 | HPV 16 F HEX | 0.06 | 8 |
| 20 | HPV 16 R HEX | 0.06 | 8 |
| 21 | HPV 18 F HEX | 0.06 | 8 |
| 22 | HPV 18 R HEX | 0.06 | 8 |

c) Final concentrations of the primers in the reaction mixture for the third ROX at—internal control—equalled:

| | | Final concentration in the | Initial concentration |
|---|---|---|---|
| No. | PRIMER | mixture [μM] | [μM] |
| 23 | HBE1 IC F | 0.2 | 8 |
| 24 | HBE1 IC R | 0.2 | 8 |

d) Final concentrations of the probes in the reaction mixture equalled:

| | | Final | Initial |
|---|---|---|---|
| | | concentration in | concentration |
| No. | PROBE | the mixture [μM] | [μM] |
| 25 | HR-HPV | 0.36 | 8 |
| 26 | 33n | 0.36 | 8 |
| 27 | 16/18 | 0.06 | 8 |
| 28 | IC | 0.2 | 3.6 |

Appropriate amounts of primers and probes at the above concentrations were prepared. Before the mixture was prepared, all ingredients were vortexed and centrifuged.

Subsequently, the mixture was divided into appropriate portions, which were placed into test tubes.

3. Description of the Master MIX MabPROBE Analysis

Real-Time Master Mix MabProbe is a 2.083× concentrated mixture for Real-Time PCR. It contains a Taq polymerase blocked with a monoclonal antibody and other ingredients required for Real-Time PCR. The ingredients of the mixture were combined in appropriate portions into the final mixture, whose use reduces the risk of contamination and human error. The kit comprises:
Master MIX MabPROBE
A mixture of probes and primers
A positive control sample
A negative control sample
Water

Composition of the Master MIX MabPROBE mixture:
Run MAb polymerase 0.104 U/μl
Reaction buffer: Tris-HCl 20.83 mM pH 8.0,
KCl 104 mM
MgCl2—10.42 mM
dNTPs—2.75 mM (0.69 mM of each dNTP)
PCR stabilizers

All components of the test should be stored at −20° C. in a dark place. Re-thawing and refreezing of components does not cause any deterioration of their activity. They must be transported on dry ice.

4. Test Protocol:

a. All ingredients should be placed on ice. Primers, probes and the Master Mix MabPROBE mixture, positive control sample and negative control sample should be vortexed and briefly centrifuged.

b. In the following table, the quantities of the ingredients for one reaction are shown. In order to perform more analyses, it is necessary to appropriately recalculate the composition of the mixture, taking into consideration test samples, positive control and negative control. The addition of a UNG enzyme in a dilution of 1:200 is optional, and it may be replaced with water. Appropriate amounts of individual components should be mixed together in a 1.5 ml test tube of the Eppendorf type, and then the resulting mixture should be vortexed and briefly centrifuged.

Composition of mixture for a single reaction in 25 μl

| Ingredients | Quantity for one reaction in 25 μl |
|---|---|
| MASTER MIX MAB | 12 |
| PRIMERS + PROBES | 6 |
| H₂O or optionally *UNG (1:200) | 1 |
| **DNA | 6 |

The UNG enzyme is not provided with the kit. It can be purchased through the Fermentas company at a concentration of 1 μl/U.

The recommended quantity of DNA matrix is 10 pg-1 μg. The maximal volume of the added DNA eluate is 6 μï. The quantity and volume of the added DNA eluate depends on the DNA isolation and is subjected to optimisation. In case the added quantities are below 6 μï, the missing quantity should be replaced with water.

c. Place 19 μl of the mixture into test tubes dedicated to the apparatus used for conducting the reaction.

d. Next, successively add to each tube 6 μï of the test DNA, positive control and negative control, repipetting several times. The mixture prepared in this way should be briefly centrifuged.

e. Place the samples into a thermal cycler.

Thermal Cycler Programme:

Time Temperature ° C. Number of cycles

UNG hydrolysis 20 minutes 37° C. 1

Denaturation 15 minutes 95° C. 1

Amplification 20 seconds 95° C. 40-50*

75 seconds 60° C. the number of cycles should be optimised

Cooling 30 seconds 37° C. 1

The test was verified on the following apparatuses:
LightCycler 2.0, LightCycler Roche 96, LightCycler Roche 480, Biorad CFXTouch. The devices, which require colour compensation, are provided by the manufacturer of the test with a file in the correct format or a kit of dyes for self-performed compensation.

5. Interpretation of the Results

According to the invention, the presence of the virus in a specific sample is evidenced by the amplification of a product comprising its specific pair of primers.

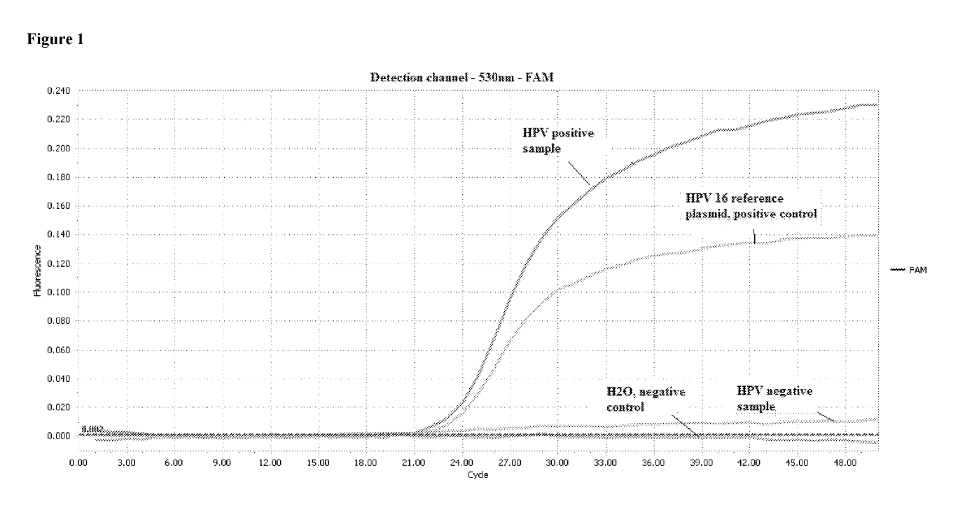

In the present embodiment, the reaction product, which indicates the presence of a HPV virus, was identified by an analysis of a logarithmic growth curve formed as a result of the observed change of fluorescence for the test sample. Positive samples were characterised by a logarithmic increase of the fluorescence level with respect to the relatively constant level of fluorescence in negative samples. Preferably, the analysis was conducted using appropriate controls: negative, positive and internal ones. The resulting image is shown in FIG. 1-3.

In FIG. 1, the results of a real-time PCR for all oncogenic test HPV types, preferably using a positive control sample, a negative and a positive control sample and a negative clinical sample, were presented. According to the embodiment of the invention, the presence or lack of oncogenic HPV in the investigated samples is read out in a detection channel of 530 nm. A logarithmic increase of the PCR product in the positive clinical sample indicates the presence of at least one from the group of all investigated oncogenic HPV types. No logarithmic growth of the PCR product in the negative clinical sample demonstrates a lack of presence of oncogenic HPVs. Preferably, a positive and a negative control sample were applied in the detection channel at 530 nm. In order to facilitate the interpretation of the results, a comparison of the control sample with clinical test samples is preferable.

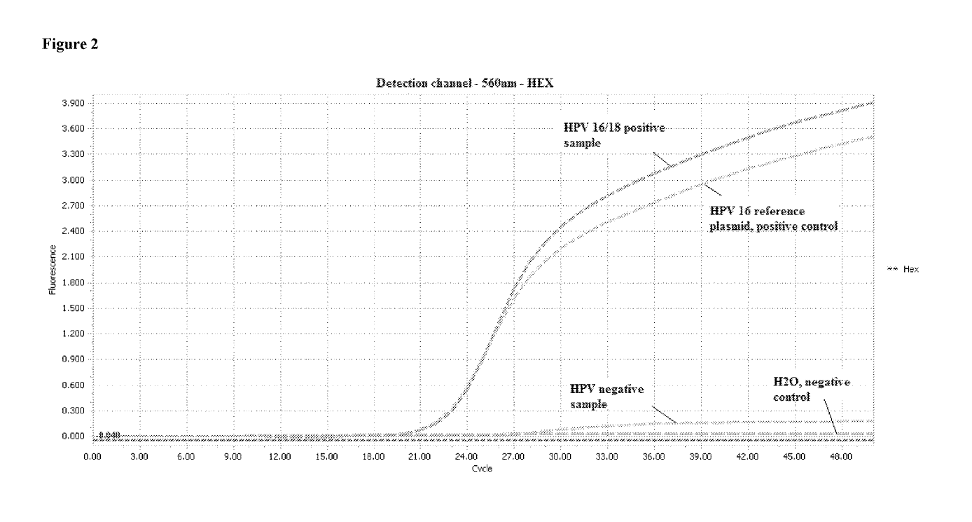

In FIG. 2, the results of a real-time PCR for 16 and 18 HPV types, preferably using a positive control sample, a negative and a positive control sample and a negative clinical sample, were presented. According to the embodiment of the invention, the presence or lack of 16 or 18 HPV types in the investigated samples is read out in a detection channel of 560 nm. A logarithmic increase of the PCR product in the positive clinical sample indicates the presence of at least one from the investigated oncogenic 16 and 18 HPV types. No logarithmic growth of the PCR product in the negative clinical sample demonstrates a lack of presence of oncogenic 16 and 18 HP Vs. Preferably, a positive and a negative control sample were applied in the detection channel at 560 nm. In order to facilitate the interpretation of the results, a comparison of the control sample with clinical test samples is preferable.

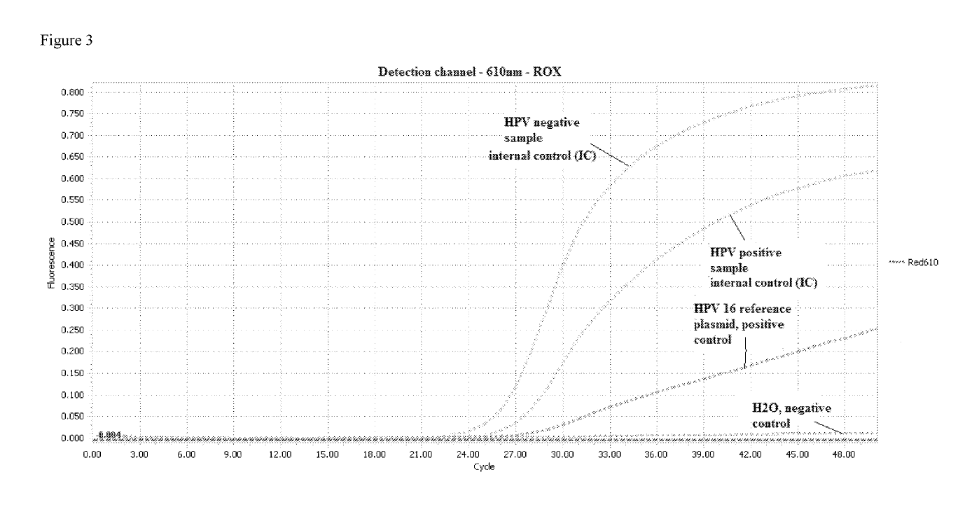

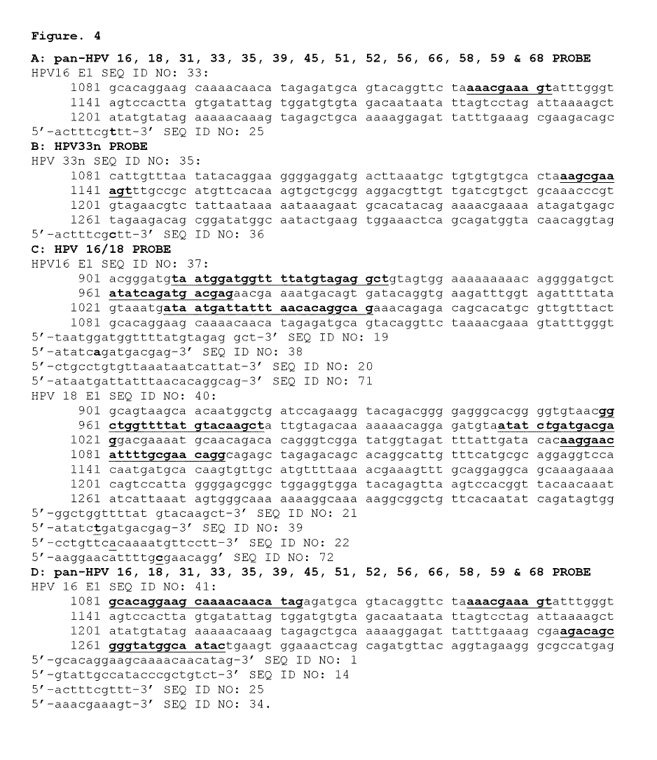

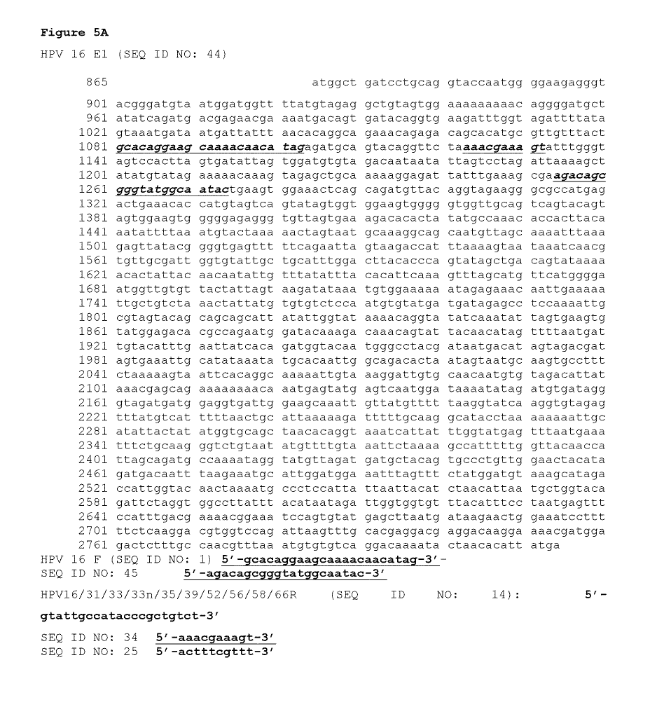

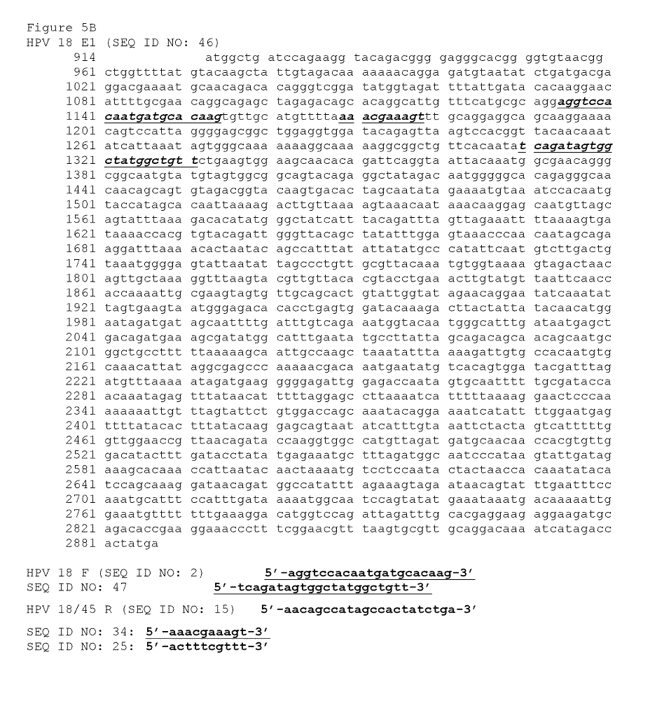

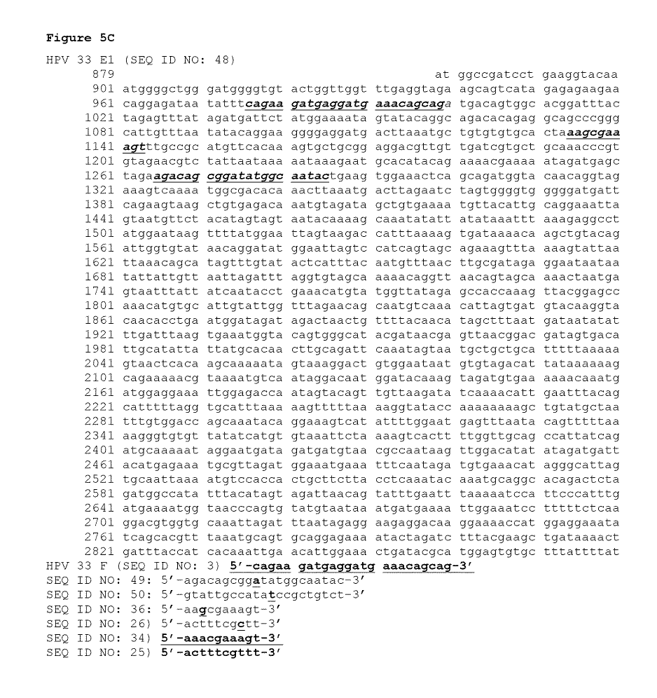

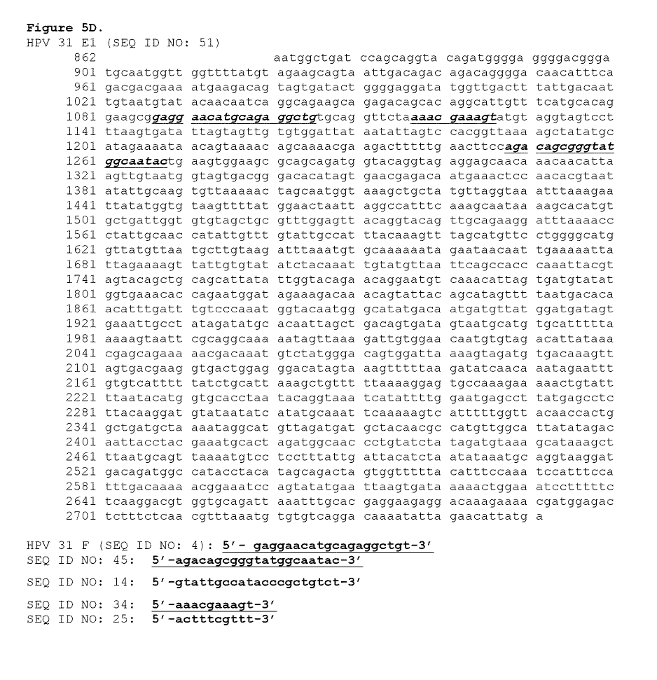

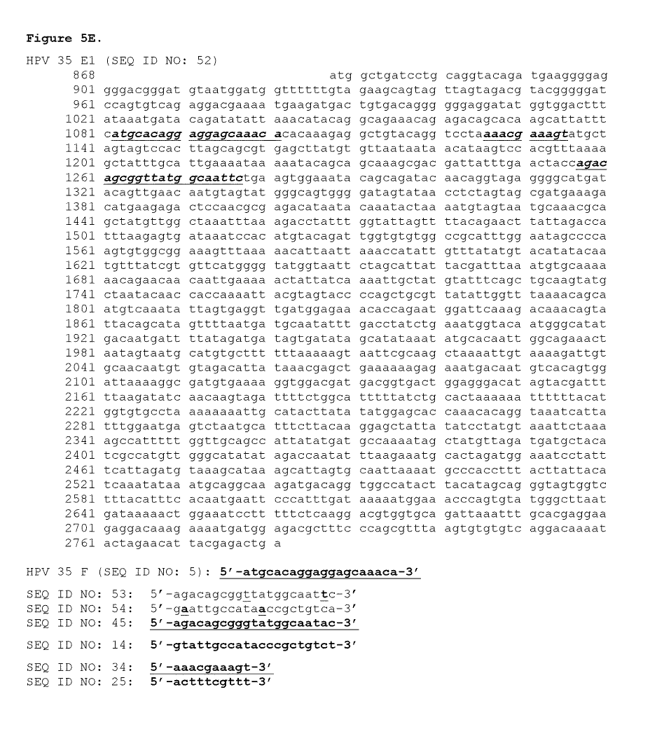

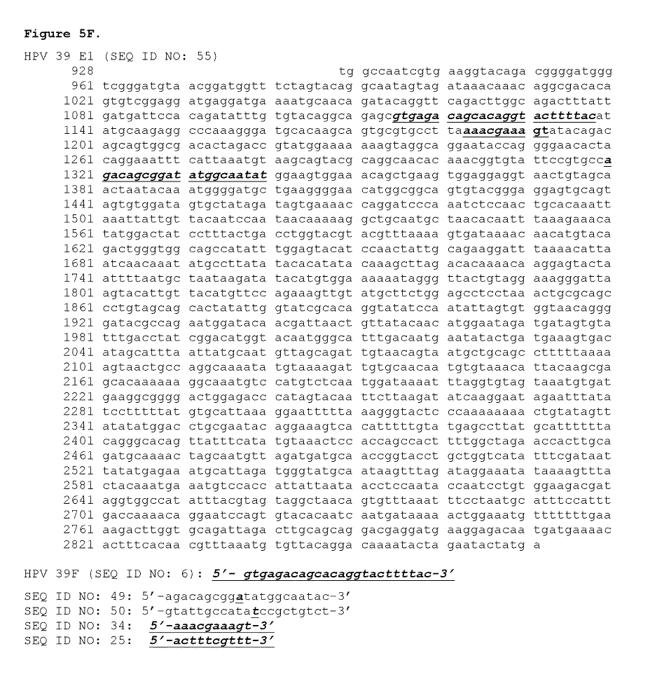

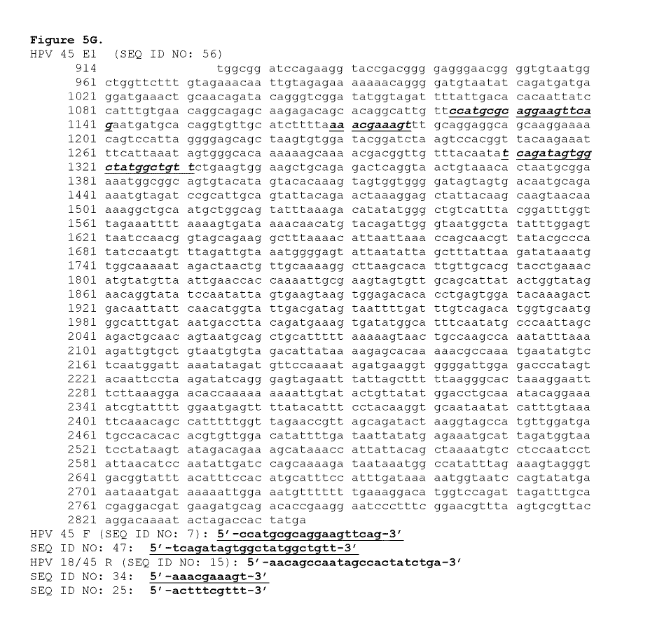

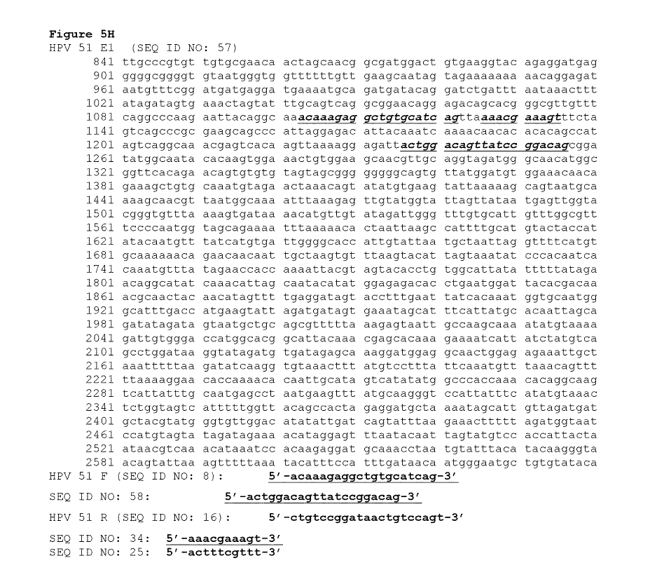

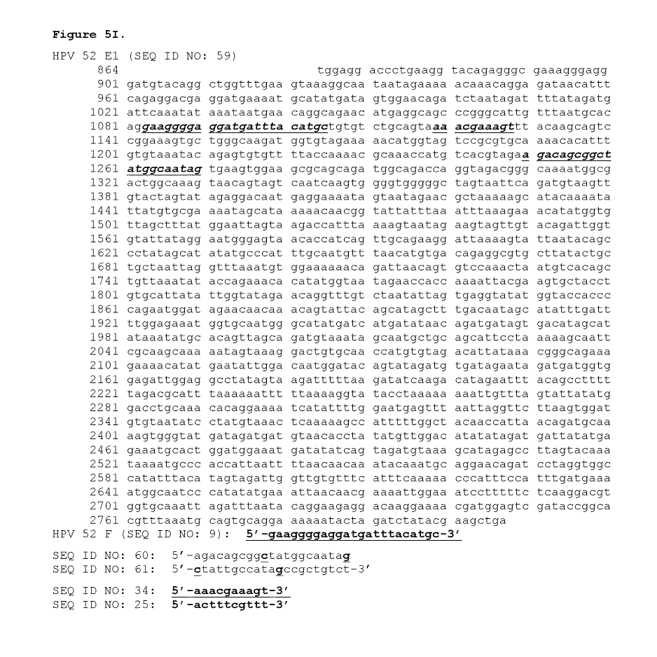

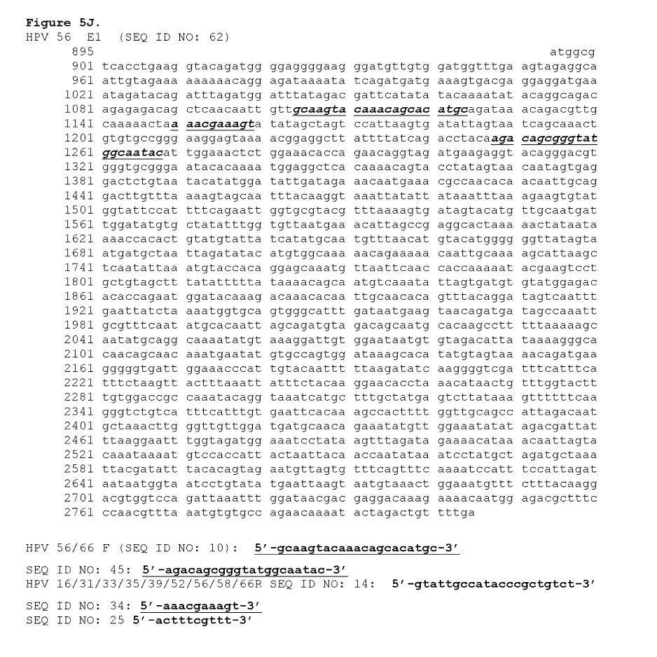

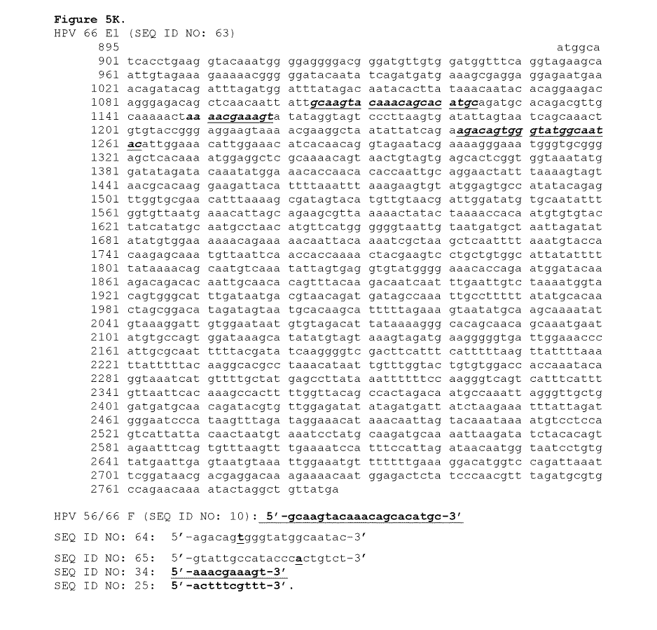

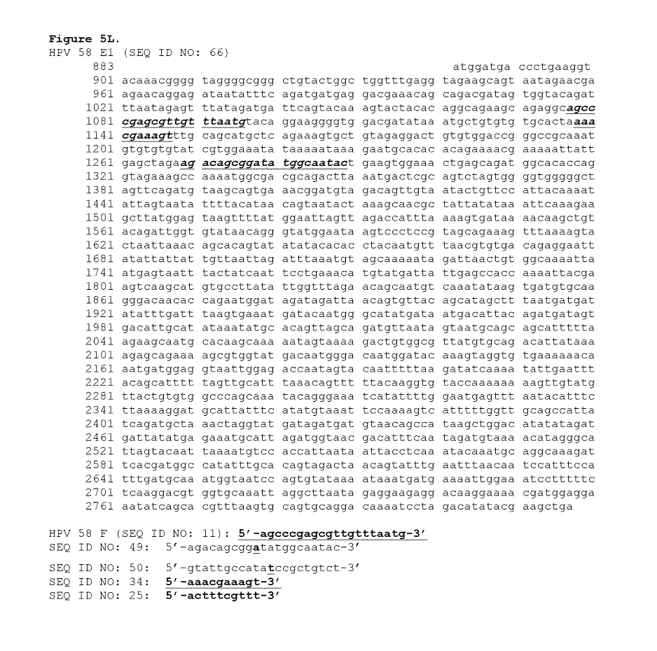

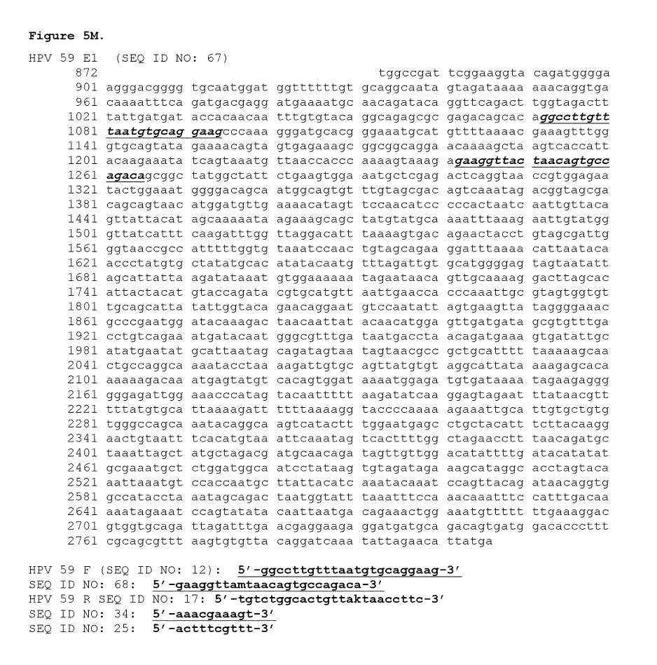

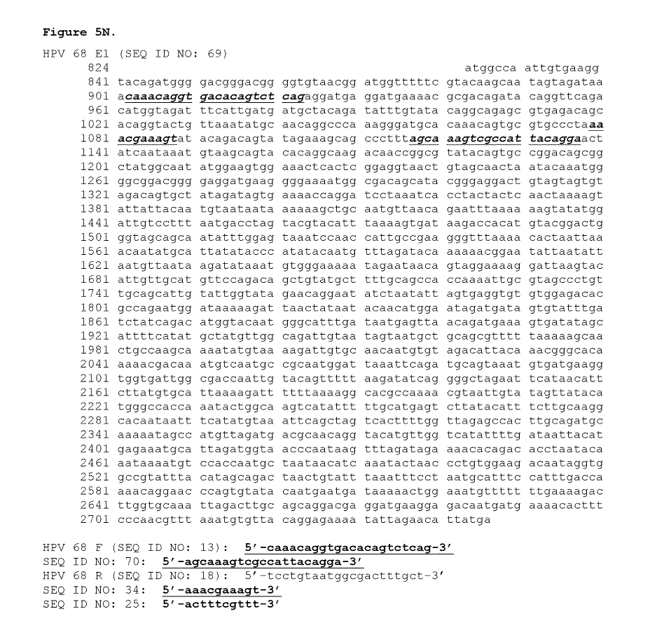

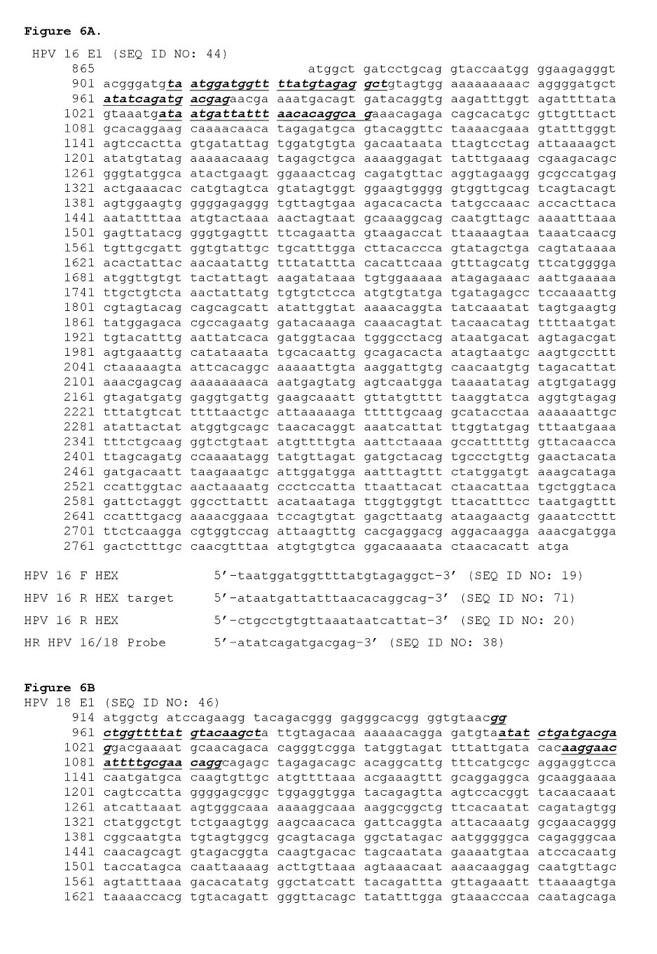

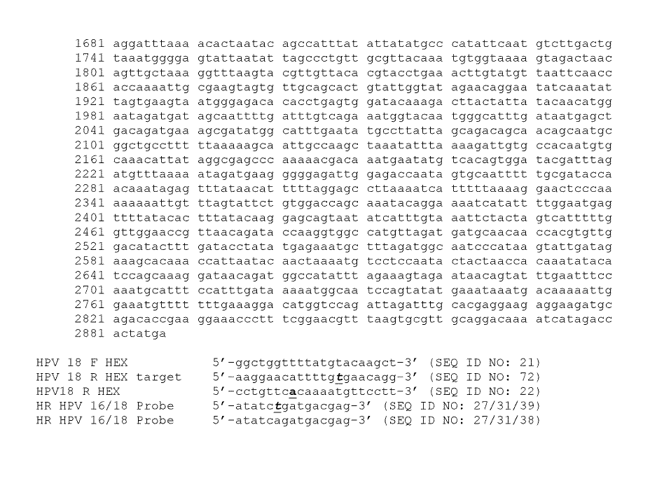

In FIG. 3, the results of a real-time PCR for the internal control (IC), preferably using a positive control sample, a negative and a positive control sample and a negative clinical sample, were presented. According to the embodiment of the invention, the presence or lack of the internal control (IC) in the investigated samples is read out in a detection channel of 610 nm. A logarithmic growth of the PCR product in the positive control sample and in the positive and negative clinical samples indicates the presence of a fragment of human beta globin and provides evidence for the correct course of the reaction and the appropriate quantity of DNA needed to detect all the investigated oncogenic HPV types. The absence of logarithmic growth of the PCR product in the negative clinical sample is expected, which suggests no presence of contamination with human DNA or the amplified PCR product. In order to facilitate the interpretation of the results, a comparison of the control sample with clinical test samples is preferable.

The present embodiment is not an example limiting the scope of the invention. The reaction product may be identified by any methods known to individuals skilled in the art.

Preferably, it can be sequenced to identify any specific sequence of the virus present in the investigated sample.

An exemplary PCR product obtained from the pair of primers HPV 16 F and HPV 16/31/33/33n/35/39/52/56/58/66R has been presented below (SEQ ID NO: 43):

HPV 16 F
5′ GC AC AGGAAGC AAAAC AAC AT AGAGATGC AGT AC
AGGTTCT AAAACGAAAGT
ATTTGGGTAGTCCACTTAGTGATATTAGTGGATGTGTAGACAATAATATT
AGTCCTAGATTAAAAGCTATATGTATAGAAAAACAAAGTAGAGCTGCAAA
AAGGAGATTATTTGAAAGCGAAGACAGCGGGTATGGCAATAC3'
HPV16/31/33/33n/35/39/52/56/58/66R

### Example II

### Evaluation of the Effectiveness of Different Kits of Primers and Molecular Probes for the Detection of Oncogenic HPV Types

The first step involved a modification of the composition of primers and molecular probes described above, so as to separate an additional detection channel for 16/18 HPVs. According to epidemiological data, HPV 16 and HPV 18 are characterised by the highest oncogenic potential and risk of development of cervical cancer in infected women. Therefore, it is recommended for HPV tests used for clinical purposes and, in particular, for population-based screening tests to allow not only for identification of an infection with oncogenic HPVs, but also for determination of the presence of HPV 16/18 in the investigated sample. Therefore, an additional kit of primers and one molecular probe allowing for discrimination of HPV 16/18 in a single reaction with a system for a group of all oncogenic HPV types and an internal control was designed. As a result of the conducted studies, 4 primers for HPV 16 and HPV 18 and one short probe TaqMan-LNA labelled with HEX fluorescent dye, allowing for the detection of amplicons in the channel of 560 nm, were obtained. The El region of the HPV genome, situated at a distance of approximately 700 bp from the consensus region for all oncogenic HPV types, was selected as a target sequence for primers and a common LNA probe. In this case, the internal control system was favourably modified for the real-time PCR. The synthetic nucleotide was eliminated in favour of a reference human gene for beta globin. 2 primers and a TaqMan probe labelled with ROX dye (detection channel at 610 nm) were designed for single-stage amplification of this gene together with HPV particles, potentially present in the sample. As a result, the target reaction mixture contained 26 primers and 4 molecular probes. As a consequence of performing the test on previously genotyped cytological samples, neither products of the primer-dimer type nor other non-specific PCR amplification products were observed.

The aim of the analysis was to confirm whether the selected configuration of primers and LNA-TaqMan probes is characterised by the highest efficiency in the detection of oncogenic HPV types in clinical cytological material. Comparative analyses of the new test comprising a potentially optimal system of primers and probes were conducted by evaluation of parameters such as sensitivity and specificity of the detection of oncogenic HPV types, the detection threshold for individual HPV types, including all oncogenic types, and the correlation of the obtained results with the clinically validated HPV PapilloCheck test (Greiner Bio-One), which is approved for clinical use with the CE-IVD certificate.

The analysis comprises 1256 pairs of the results of the new test for the presence of oncogenic HPV types (hrHPV) and validated PapilloCheck test. The material was collected from women, residents of the Zachodniopomorskie province and Wielkopolska province, who presented for routine preventive cytological exams, including 720 (57.3%) patients with normal cytological results and 536 (42.7%) selected women whose cytological exam revealed the presence of abnormal epithelial cells, belonging to diagnostic categories ASC-US (n=249; 19.8%), ASC-H (n=20; 1.6%), L-SIL (n=232; 18.5%) and H-SIL (n=34; 2.7%) and L-SIL/H-SIL (n=l; 0.1%). The average age of the investigated women equalled 33.4±9.6 years of age (range: 16-66 years of age). DNA for the real-time PCR was isolated from cytology samples (cervical smears) collected into a SurePath liquid medium (Becton Dickinson), using a Cervex Brush Combi brush (Rovers). DNA isolation was performed automatically using magnetic beads in a NucliSense EasyMAG apparatus (Biomerieux). DNA amplification was performed in a Light Cycler 96 apparatus (Roche). Thin-layer cytological slides, prepared using a PrepStain robot (Becton Dickinson), were evaluated by an experienced team of cytomorphologists and pathologists using computer-assisted screening (Focal Point GS system, Becton Dickinson). The results of cytological and molecular genetic exams carried out using hrHPV and PapilloCheck tests were subjected to a statistical analysis. The detection threshold for selected HPVs (a minimal number of copies of viral particles/genomes) in the designed hrHPV test was determined. For this purpose, HPV16 and HPV18 reference materials with a known number of copies of the mentioned viruses (1st WHO International Standard for Human Papillomavirus, National Institute for Biological Standards and Control, UK) were purchased.

11 serial dilutions of HP VI 6 and HPV18 standards were performed to determine standard curves in three series of repetitions in order to define the following detection thresholds using real-time PCR:

HPV16 standard: HR-HPV (FAM) channel—control dilution 1: 128−2×104 copies of the virus

16/18 HPV channel (HEX)—control dilution 1: 2048−3.33×103 copies of the virus

HPV18 standard: HR-HPV channel (FAM)—control dilution 1: 128−5×104 copies of the virus

16/18 HPV channel (HEX)—control dilution 1: 2048−1.92×103 copies of the virus

It is worth noting that the detection threshold for 16/18 HPVs in the second detection channel is lower in comparison to the group of all oncogenic HPVs in the first detection channel, which seems to be a favourable phenomenon in terms of the clinical efficacy of the test (targeting HPV types with a higher oncogenic potential). It should also be emphasized that taking into account the above-described correlation coefficients of the new test and the reference PapilloCheck test, it can be concluded that the detection threshold of oncogenic HPV types is similar for both tests.

Possible cross-reactions of the components of the hrHPV test with other microorganisms were also assessed. For this purpose, based on a review of the available literature (including: P. Gajer et al. Temporal dynamics of the Human vaginal microbiota, Science Translational Medicine 2012, 4, 132ra52; S. S. Witkin, W. J. Ledger: Complexities of the uniquely human vagina. Science Translational Medicine 2012, 4, 132fsl l), 42 bacterial strains, typical physiological inhabitants of the reproductive tract in women, and pathogens causing the most common infections of the genitourinary system were selected. Reference strains of the selected bacteria were purchased in the ATCC: The Global Bioresource Center, and they comprised: Escherichia coli ATCC 11775, Escherichia coli ATCC 8739, Proteus vulgaris ATCC 6380, Proteus mirabilis ATCC 7002, Staphylococcus epidermidis ATCC 49461, Streptococcus agalactiae (B) ATCC 12386, Listeria monocytogenes (4e) ATCC 19118, Listeria monocytogenes (4e) ATCC 19118, Listeria innocua (6a) ATCC 33090, Lactobacillus gasseri ATCC 19992, Lactococcus lactis ATCC 19435, Lactobacillus acidophilus ATCC 4356, Candida albicans ATCC 10231, Corynebacterium urealyticum ATCC 43044, Oligella urethralis ATCC 17960, Aerococcus viridans ATCC 700406, Neisseria gonorrhoeae ATCC 49226, Gardnerella vaginalis ATCC 14018, Veillonella parvula ATCC 10790, Fusobacterium nucleatum subsp. nucleatum ATCC 25586, Gemella morbillorum ATCC 27824, Actinomyces odontolyticus ATCC 17929, Peptostreptococcus anaerobius ATCC 27337, Parvimonas micra ATCC 33270, Ureaplasma parvum ATCC 27813, Peptoniphilus asaccharolyticus ATCC 14963, Finegoldia magna ATCC 29328, Porphyromonas levii ATCC 29147, Prevotella loescheii ATCC 15930.

In the course of the performed experiments, no cross-reaction between the designed system of primers and probes and the above-mentioned microorganisms was obtained in the hrHPV test.

To conclude: the study resulted in the development of an optimal kit of primers and reaction mixture favourable for a single-stage amplification of oncogenic HPV types, HPV 16/18 and an internal control in three detection channels.

Comparative studies of the correlation of the new test with the results of the reference test (PapilloCheck) in the group of 1256 women were performed for the purpose of further statistical analysis.

A clinically desired detection threshold for HPV 16/18 and other oncogenic HPV types similar to the reference test was obtained.

No cross-reactions of the new test with other microorganisms present in the reproductive tract of women were observed, which proves the high specificity of the test.

### Example III

### Results Analysis and Statistics for the Effectiveness of the New HR-HPV Test for Detecting the Presence of Oncogenic HPV Viruses

In order to determine the diagnostic value of the new test for HR-HPV, its sensitivity and specificity in relation to the validated PapilloCheck test (Greiner Bio-One) were measured. It was assumed that the latter test is characterised by an optimal diagnostic accuracy for HR-HPV infections, including HPV 16/18, which is well described in the literature. Therefore, it was used as a reference for the new test. In addition, correlation coefficients for both tests were determined. This is a standard procedure to apply in the case of validation of qualitative tests, which allows for determining whether the currently used gold standard could be replaced with a new tool. The analysis was carried out on the basis of the results of cytological and molecular genetic exams, obtained by the NZOZ Meditest.

The level of correlation between the results of the new tests and the results of the reference test was evaluated based on the value of Kendall's tau b correlation coefficient. The sensitivity and specificity of the new test compared to the reference test, understood as the percentage of the results of true positives and true negatives, respectively, were assessed on the basis of ROC analysis. All statistical calculations were performed using the Statistica 10 software package (StatSoft, Tulsa, Okla., United States).

Detection of HR-HPV in the Entire Investigated Group

A total of the results of 1256 pairs of tests was analysed. The new test produced 630 (50.2%) positive results and 626 (49.8%) negative results. The reference test produced 647 (51.5%)) positive results and 609 (48.5%>) negative results.

| | Reference test - | Reference test - | |
|---|---|---|---|
| Test/result | positive result | negative result | Total |
| New test - | 612 | 18 | 630 |
| positive result | | | |
| New test - | 35 | 591 | 626 |
| negative result | | | |
| Total | 647 | 609 | 1256 |

In the case of 18 (2.9%) positive results of the new test, the reference test produced a negative result. In the case of 35 (5.6%>) negative results of the new test, the reference test produced a positive result. Kendall's tau b correlation coefficient equalled for both tests b=0.9159327.

ROC analysis showed that the new test is characterised by 94.6% sensitivity and 97.0%) specificity compared to the reference test.

Detection of HPV 16/18 in the entire investigated group

A total of the results of 1256 pairs of tests was analysed. The new test produced 230 (18.3% o) positive results and 1026 (81.7%) negative results. The reference test produced 238 (18.9% o) positive results and 1018 (81.1%) negative results.

Test/result Reference test Reference test Total

positive result negative result

New test—positive 223 7 230

result

New test—negative 15 101 1 1026

result

Total 238 1018 1256

In the case of 7 (3.0%) positive results of the new test, the reference test produced a negative result. In the case of 15 (1.5%) negative results of the new test, the reference test produced a positive result. Kendall's tau b correlation coefficient for both tests equalled b=0.9424401.

ROC analysis showed that the new test is characterised by 93.7% sensitivity and 99.3% specificity compared to the reference test.

Detection of HR-HPV in the Group with Normal Cytology

A total of the results of 720 pairs of tests was analysed. The new test produced 154 (21.4%)) positive results and 566 (78.6%) negative results. The reference test produced 165(22.9%)) positive results and 555(77.1%) negative results.

| | Reference test - | Reference test - | |
|---|---|---|---|
| Test/result | positive result | negative result | Total |
| New test - positive result | 145 | 9 | 154 |
| New test - negative | 20 | 546 | 566 |
| result | | | |
| Total | 165 | 555 | 720 |

In the case of 9 (5.8%) positive results of the new test, the reference test produced a negative result. In the case of 20 (3.5%) negative results of the new test, the reference test produced a positive result. Kendall's tau b correlation coefficient for both tests equalled b=0.8841279.

ROC analysis showed that the new test is characterised by 87.9% sensitivity and 98.4%) specificity compared to the reference test.

Detection of HPV 16/18 in the group with normal cytology

A total of the results of 720 pairs of tests was analysed. The new test produced 52 (7.2%)) positive results and 668 (92.8%>) negative results. The reference test produced 53 (7.4% o) positive results and 667 (92.6%>) negative results.

| | Reference test - | Reference test - | |
|---|---|---|---|
| Test/result | positive result | negative result | Total |
| New test - | 49 | 3 | 52 |
| positive result | | | |
| New test - | 4 | 664 | 668 |
| negative result | | | |
| Total | 53 | 667 | 720 |

In the case of 3 (5.8%>) positive results of the new test, the reference test produced a negative result. In the case of 4 (0.6%>) negative results of the new test, the reference test produced a positive result. Kendall's tau b correlation coefficient for both tests equalled b=0.9281394.

ROC analysis showed that the new test is characterised by 92.5% sensitivity and 99.6%) specificity compared to the reference test.

Detection of HR-HPV in the Group with Abnormal Cytology (Minimum ASC-US)

A total of the results of 536 pairs of tests was analysed. The new test produced 476 (88.8% o) positive results and 60 (11.2%>) negative results. The reference test produced 482 (89.9% o) positive results and 54 (10.1%>) negative results.

Test/result Reference test Reference test Total

positive result negative result

New test—positive 467 9 476

result

New test—negative 15 45 60

result

Total 482 54 536

In the case of 9 (1.9%) positive results of the new test, the reference test produced a negative result. In the case of 15 (25.0%) negative results of the new test, the reference test produced a positive result. Kendall's tau b correlation coefficient for both tests equalled b=0.7658275.

ROC analysis showed that the new test is characterised by 96.9% sensitivity and 83.3%) specificity compared to the reference test.

Detection of HPV 16/18 in the Group with Abnormal Cytology (Minimum ASC-US)

A total of the results of 536 pairs of tests was analysed. The new test produced 178 (33.2%)) positive results and 358 (66.8%>) negative results. The reference test produced 185 (34.5%)) positive results and 351 (65.5%>) negative results.

| | Reference test - | Reference test - | |
|---|---|---|---|
| Test/result | positive result | negative result | Total |
| New test - positive result | 174 | 4 | 178 |
| New test - negative | 11 | 347 | 358 |
| result | | | |
| Total | 185 | 351 | 536 |

In the case of 4 (2.2%) positive results of the new test, the reference test produced a negative result. In the case of 11 (3.1%>) negative results of the new test, the reference test produced a positive result. Kendall's tau b correlation coefficient for both tests equalled b=0.9379317.

ROC analysis showed that the new test is characterised by 94.1% sensitivity and 98.9%) specificity compared to the reference test.

### CONCLUSIONS

Analysis of Kendall's tau b coefficients revealed that the results of the new test are characterised by a very high correlation with the results of the reference PapilloCheck test within the scope of HR-HPV detection, ranging from 76.6% to 91.5%. An even higher level of correlation, close to the ideal threshold of 95% (from 92.8%> to 94.2%>), was obtained for the detection of HPV 16/18.

The high diagnostic accuracy of the new test is confirmed by its high sensitivity (from 87.9% to 96.9% for hrHPV and from 92.5% to 94.1% for HPV 16/18) and specificity (from 83.3% to 98.4% for HR-HPV and from 98.9% to 99.3% for HPV 16/18). The very high specificity of the new test for the detection of HPV 16/18 deserves particular attention; the obtained values prove that a negative result of the test excludes the presence of HPV 16 or HPV 18 infection with a probability of almost 100%. The fact that a high level of correlation, sensitivity and specificity of the new test was observed in both subgroups of patients with normal and abnormal results of cytological exam confirms the versatility of this tool.

To conclude: As a result of the performed investigations, a very high correlation of the results of the hrHPV test, according to the invention, with the results of the well-validated clinically commercial reference test (PapilloCheck) was obtained, and the high sensitivity and specificity of the hrHPV test, according to the invention, in relation to the reference test (PapilloCheck) was confirmed.

Determination of the clinical effectiveness of the new HR-HPV test for detecting precancerous lesions and cervical cancer

A clinical population-based study, which involved 5000 women, using the new hrHPV test in cytology samples from the cervix collected on a SurePath liquid medium for further statistical analyses of correlation with a microscope image and determination of the clinical effectiveness of the new test was conducted.

The analysis was aimed at determining the clinical usefulness of the new hrHPV test in preventive exams and diagnosis of precancerous lesions and cervical cancer. To determine the diagnostic effectiveness of the new test, a large group of women who presented for routine cytological exams, being a part of cervical cancer prophylaxis, was investigated. The correlation of the obtained molecular genetic results with the microscopic image of cytological slides in a liquid medium, prepared using the standardised SurePath method of the Becton Dickinson company and assessed by qualified cytomorphologists and pathologists according to the Bethesda classification system of 2001, was determined. In order to obtain higher objectification of the microscopic evaluation, the slides were further evaluated using computer-assessed automatic screening (FocalPoint system of the Becton Dickinson company).

The analysis included 5000 pairs of the new test results for the presence of HR-HPV and the results of cytological exams on a liquid medium. The material was collected from consecutive women from the Zachodniopomorskie province and Wielkopolska province who presented for routine cytological exams. Patients whose tests for the presence of HR-HPV were ordered by the attending physician and women with a history of gynaecological and oncological abnormalities were excluded from the analysis. The average age of the investigated women equalled 33.6±10.1 years of age (range: 16-67 years of age). The results of the cytological exam comprised 4825 (96.5%) normal preparations (no abnormal epithelial cells), 77 (1.55%) preparations with lesions corresponding to ASC-US, 6 (0.1%) preparations with lesions corresponding to ASC-H, 77 (1.55%) preparations with lesions corresponding to L-SIL and 15 (0.3%) preparations with lesions corresponding to H-SIL.

Similarly to the described above, DNA for the real-time PCR was isolated from cytology samples (cervical smears) collected into a SurePath liquid medium (Becton Dickinson), using a Cervex Brush Combi brush (Rovers). DNA isolation was performed automatically using magnetic beads in a NucliSense EasyMAG apparatus (Biomerieux). PCR was prepared using a PIRO automated pipetting station (Dornier LTF). DNA amplification was performed in a Light Cycler 96 apparatus (Roche). As mentioned above, thin-layer cytological slides, prepared using a PrepStain robot (Becton Dickinson), were evaluated by an experienced team of cytomorphologists and pathologists using computer-assisted screening (Focal Point GS system, Becton Dickinson). The results of cytological and molecular genetic exams were subjected to a statistical analysis.

Results analysis and statistics of the clinical effectiveness of the new HR-HPV test for detecting precancerous lesions and cervical cancer

In order to determine the clinical usefulness of the new test for HR-HPV, the values of the positive (PPV) and negative (NPV) predictive value in relation to the cytological exam in a liquid medium (SurePath, Becton Dickinson) were used. Thus, the probability of obtaining an abnormal result of a cytological exam in patients having a positive result of the new test and a normal result of a cytological exam with a negative result of the test for the presence of HR-HPV was determined. In case a high negative predictive value of the new test is confirmed, its negative result would permit resignation from a cytological exam, and its positive result could be a criterion for its execution.

The level of correlation between the results of the new hrHPV test and the results of the cytological exam was evaluated based on the value of Kendall's tau b correlation coefficient. The positive predictive value (PPV, the probability of an abnormal result of a cytological exam in an individual with a positive result of the test for the presence of HR-HPV) and negative predictive value (NPV, the probability of a normal result of a cytological exam in an individual with a negative result of the test for the presence of HR-HPV) were evaluated based on ROC analysis. All statistical calculations were performed using the Statistica 10 software package (StatSoft, Tulsa, Okla., United States).

HR-HPV Detection Versus an Abnormal Result of the Cytological Exam (ASCUS Minimum)

A total of the results of 5000 pairs of hrHPV tests and cytological exams was analysed. The new test produced 403 (8.1%) positive results and 4597 (91.9%) negative results. The results of the cytological exam comprised 175 (3.5%) abnormal slides and 4825 (96.5%) normal slides.

| | Abnormal cytology (min. | | |
|---|---|---|---|
| Test/result | ASCUS) | Normal cytology | Total |
| New test - positive | 168 | 235 | 403 |
| result | | | |
| New test - negative | 7 | 4590 | 4597 |
| result | | | |
| Total | 175 | 4825 | 5000 |

In the case of 235 (58.3%) patients with a positive result of the new test, the cytological exam produced a normal result. In the case of 7 (0.2%) patients with a negative result of the new test, the cytological exam produced an abnormal result. Kendall's tau b correlation coefficient for the test for HR-HPV presence and the result of a cytological exam equalled b=0.6152294.

ROC analysis showed that the new test is characterised by a positive predictive value (PPV) of 41.7%) and a negative predictive value (NPV) of 99.8%, compared to the results of a cytological exam.

HR-HPV Detection Versus an Abnormal Result of the Cytological Exam (H-SIL)

A total of the results of 5000 pairs of hrHPV tests and cytological exams was analysed. The new test produced 403 (8.1%>) positive results and 4597 (91.9%) negative results. The results of the cytological exam comprised 15 (0.3%) slides with lesions corresponding to H-SIL and 4985 (99.7%)) normal slides or slides with intensity lower than H-SIL.

| | Abnormal cytology | | |
|---|---|---|---|
| Test/result | (H-SIL) | Normal cytology | Total |
| New test - positive | 15 | 388 | 403 |
| result | | | |
| New test - | 0 | 4597 | 4597 |
| negative result | | | |
| Total | 15 | 4985 | 5000 |

In the case of 388 (96.3%) patients with a positive result of the new test, the cytological exam revealed no lesions corresponding to H-SIL. In patients with a negative result of the new test, the cytological exam revealed no lesions corresponding to H-SIL. Kendall's tau b correlation coefficient for the test for HR-HPV presence and the result of a cytological exam equalled b=0.1852669.

ROC analysis showed that the new test is characterised by a positive predictive value (PPV) of 3.7%) and a negative predictive value (NPV) of 100.0%, compared to the results of a cytological exam.

HR-HPV 16/18 Detection Versus an Abnormal Result of the Cytological Exam (ASCUS Minimum)

A total of the results of 5000 pairs of tests and cytological exams was analysed. The new test produced 169 (3.4%) positive results and 4831 (96.6%) negative results. The results of the cytological exam comprised 175 (3.5%) abnormal slides and 4825 (96.5%) normal slides.

| | Abnormal cytology | Normal | |
|---|---|---|---|
| Test/result | (min. ASCUS) | cytology | Total |
| New test - positive result | 71 | 98 | 169 |
| New test - negative result | 104 | 4727 | 4831 |
| Total | 175 | 4825 | 5000 |

In the case of 98 (58.0%) patients with a positive result of the new test, the cytological exam produced a normal result. In the case of 104 (2.2%) patients with a negative result of the new test, the cytological exam produced an abnormal result. Kendall's tau b correlation coefficient for the test for HPV 16/18 presence and the result of a cytological exam equalled b=0.3919416.

ROC analysis showed that the new test is characterised by a positive predictive value (PPV) of 42.0% and a negative predictive value (NPV) of 97.8%, compared to the results of a cytological exam.

HPV 16/18 Detection Versus an Abnormal Result of the Cytological Exam (H-SIL)

A total of the results of 5000 pairs of tests and cytological exams was analysed. The new test produced 169 (3.4%) positive results and 4831 (96.6%) negative results. The results of the cytological exam comprised 15 (0.3%) slides with lesions corresponding to H-SIL and 4985 (99.7%)) normal slides or slides with intensity lower than H-SIL.

Test/result Abnormal cytology Normal cytology or Total

(H-SIL) H-SIL(−)

New test—positive 10 159 169

result

New test—negative 5 4826 4831

result

Total 15 4985 5000

In the case of 159 (94.1%) patients with a positive result of the new test, the cytological exam revealed no lesions corresponding to H-SIL. In 5 (0.1%) patients with a negative result of the new test, the cytological exam revealed lesions corresponding to H-SIL. Kendall's tau b correlation coefficient for the test for HR-HPV presence and the result of a cytological exam equalled b=0.1921027.

ROC analysis showed that the new test is characterised by a positive predictive value (PPV) of 5.9% and a negative predictive value (NPV) of 99.9%, compared to the results of a cytological exam.

### Conclusions

The analysis of the clinical value of the new test revealed that it is characterised by an optimal negative predictive value (from 97.8% to 100%), compared to the result of the cytological exam on a liquid medium. Particularly high NPV values (100% for the negative result for HR-HPV and 99.9% for the negative result for HPV 16/18) were obtained with respect to precancerous lesions (H-SIL). The lower level of the positive predictive value is fully understandable given that not every cytological abnormality must be accompanied by an active HPV infection, especially in the group of cytological diagnoses of ASC-US and ASC-H types. It is expected that even more favourable coefficients of the predictive value for the hrHPV test can be achieved compared to traditional cytology, which is characterised by proven lesser effectiveness in detecting abnormal cervical epithelial cells in comparison to cytological methods on a liquid medium (liquid based cytology, LBC).

The obtained results indicate that in the case of a negative result of the new test, there is no need of a cytological exam. Therefore, the use of the new test could potentially allow for resignation from a cytological examination in a large population of HPV-negative women, which would enable significant savings.

However, due to the lower positive predictive value of the test, each case of its positive result should be an indication for cytology. In this context, another advantage of the new test should be emphasised: the fact that it is performed on the same material which is used for a cytological exam on a liquid medium. Accordingly, in the case of a positive result of the hrHPV test, the cytological exam can be performed using the same material, with no need for collection of additional smears from the patient. This significantly shortens the time of the diagnostic procedure and significantly reduces its cost.

To conclude: based on the results of the performed investigations, it was found that the hrHPV test, according to the invention, is characterised by an optimal negative predictive value (from 97.8% to 100%) in comparison with the results of a cytological exam on a liquid medium (SurePath), and the use of the new hrHPV test with high clinical effectiveness can potentially allow for resignation from a cytological exam in a large population of HR-HPV-negative women.

The invention allows for an innovative diagnostic tool, which is a test combining features of cytological and HPV exams. The implementation of the test according to the invention into preventive programmes should significantly increase the effectiveness of the screening for cervical cancer and rationalise the costs of further diagnosis and treatment. Furthermore, the use of a simple HPV test, preferably based on the real-time PCR technique in the material from the cytology on a liquid medium (LBC), will potentially allow for a greater automation and capacity of the diagnostic process in laboratories participating in preventive programmes. The application of the test will enable determination of the risk of cervical cancer and selection of adequate treatment. In addition, the innovative use of LNA technology, which had not been previously used in HPV diagnostics, allows for common access to the diagnostics at the highest clinical level.

1-38. (canceled)

39. A method of determining whether a sample tests positive for oncogenic human papillomavirus (HPV), the method comprising:
combining the sample with primers for real-time polymerase chain reaction (PCR) amplification of a target region of oncogenic HPV genomes, and a labelled pan-HPV probe capable of emitting a signal upon amplification of the target region,
providing conditions for real-time PCR, and
measuring for a signal emitted from the labelled pan-HPV probe,
wherein detection of the signal indicates that the sample tests positive for oncogenic HPV,
characterised in that the pan-HPV probe is a labelled locked nucleic acid (LNA) molecule comprising sequence ACTTTCGTTT (SEQ ID NO: 25), or the reverse complement thereof.

40. The method according to claim 39, wherein the pan-HPV probe is a labelled 10-nucleotide molecule of sequence consisting of ACTTTCGTTT (SEQ ID NO: 25), or the reverse complement thereof and/or the pan-HPV probe is labelled at its 5′ end with a fluorophore and at its 3′ end with a quencher, and wherein the signal emitted upon amplification of the target region is fluorescence.

41. The method according to claim 39, wherein the primers are for amplification of a target region of any of HPV16, HPV18, HPV31, HPV33, HPV35, HPV39, HPV45, HPV51, HPV52, HPV56, HPV58, HPV59, HPV66 or HPV68 or/and comprising combining the sample with a combination of primers for amplification of a target region of all of HPV16, HPV18, HPV31, HPV33, HPV35, HPV39, HPV45, HPV51, HPV52, HPV56, HPV58, HPV59, HPV66 or HPV68.

42. The method according to claim 39, wherein the primers comprise one or more of the following primer pairs:
a) for HPV16 SEQ ID NO: 1 and SEQ ID NO: 14
b) for HPV18 SEQ ID NO: 2 and SEQ ID NO: 15
c) for HPV33/33n SEQ ID NO: 3 and SEQ ID NO: 14
d) for HPV31 SEQ ID NO: 4 and SEQ ID NO: 14
e) for HPV35 SEQ ID NO: 5 and SEQ ID NO: 14
f) for HPV39 SEQ ID NO: 6 and SEQ ID NO: 14
g) for HPV45 SEQ ID NO: 7 and SEQ ID NO: 15
h) for HPV51 SEQ ID NO: 8 and SEQ ID NO: 16
i) for HPV52 SEQ ID NO: 9 and SEQ ID NO: 14
j) for HPV56/66 SEQ ID NO: 10 and SEQ ID NO: 14
k) for HPV58 SEQ ID NO: 11 and SEQ ID NO: 14
l) for HPV59 SEQ ID NO: 12 and SEQ ID NO: 17
m) for HPV68 SEQ ID NO: 13 and SEQ ID NO: 18
or/and wherein the primers comprise all of the primer pairs (a) to (m).

43. The method according to claim 39, comprising
combining the sample with primers for amplification of a target region of HPV16, HPV18, HPV31, HPV33, HPV35, HPV39, HPV45, HPV51, HPV52, HPV56, HPV58, HPV59, HPV66 and HPV68,
detecting a signal from the labelled pan-HPV probe, and
concluding that the sample tests positive for HPV16, HPV18, HPV31, HPV33, HPY35, HPY39, HPV45, HPY51, HPY52, HPY56, HPV58, HPY59, HPV66 or HPV68.

44. The method according to claim 39, comprising
combining the sample with primers for amplification of a target region of HPV16, HPY18, HPV31, HPV33, HPV35, HPV39, HPV45, HPV51, HPV52, HPV56, HPV58, HPV59, HPV66 and HPV68,
detecting no signal from the labelled pan-HPV probe, and
concluding that the sample tests negative for HPV16, HPV18, HPV31, HPV33, HPV35, HPV39, HPV45, HPV51, HPV52, HPV56, HPV58, HPV59, HPV66 and HPV68.

45. The method according to claim 39, further comprising
combining the sample with primers for amplification of a target region of HPV33n, and a labelled 33n probe capable of emitting a signal upon amplification of the target region, wherein the 33n probe and the pan-HPV probe carry the same label,
providing conditions for real-time PCR, and
measuring for a signal emitted from the labelled 33n probe or the pan-HPV probe,
wherein detection of the signal indicates that the sample tests positive for oncogenic HPV,
characterised in that the 33n probe is a labelled locked nucleic acid (LNA) molecule comprising sequence ACTTTCGCTT (SEQ ID NO: 26), or the reverse complement thereof or/and the 33n probe is a labelled 10-nucleotide molecule of sequence consisting of ACTTTCGCTT (SEQ ID NO: 26), or the reverse complement thereof.

46. The method according to claim 45, wherein the pan-HPV probe and 33n probe are labelled at the 5′ end with a fluorophore and at the 3′ end with a quencher, and wherein the signal emitted upon amplification of their respective target regions is fluorescence of the same wavelength.

47. The method according to claim 39, further comprising determining whether the sample tests positive for HPVI6 or HPVI8, the method comprising
combining the sample with primers for real time PCR amplification of a target region of HPV16 and HPV18 genomes, and a labelled 16/18 probe capable of emitting a signal upon amplification of the target region,
providing conditions for real time PCR, and
measuring for a signal from the labelled 16/18 probe,
wherein detection of a signal indicates that the sample tests positive for HPVI6 or HPV18,
characterised in that the 16/18 probe is a labelled locked nucleic acid (LNA) molecule comprising sequence ATATCWGATGACGAG (SEQ ID NO: 27).

48. The method of determining whether a sample tests positive for oncogenic human papillomavirus (HPV) HPV16 and/or HPV18, the method comprising
combining the sample with primers for real-time PCR amplification of a target region of HPV 16 and HPV 18 genomes, and a labelled 16/18 probe capable of emitting a signal upon amplification of the target region,
providing conditions for real-time PCR, and
measuring for a signal from the labelled 16/18 probe,
wherein detection of a signal indicates that the sample tests positive for HPVI6 or HPV18,
characterised in that the 16/18 probe is a labelled locked nucleic acid (LNA) molecule comprising sequence ATATCWGATGACGAG (SEQ ID NO: 27), or the reverse complement thereof.

49. The method according to claim 47, wherein the 16/18 probe is a labelled 15-nucleotide molecule of sequence consisting of ATATCWGATGACGAG (SEQ ID NO: 27), or the reverse complement thereof.

50. The method according to claim 47, wherein the 16/18 probe carries a different label from the pan-HPV probe.

51. The method according to claim 47, wherein the 16/18 probe is labelled at its 5′ end with a fluorophore and at its 3′ end with a quencher, and wherein the signal emitted upon amplification of the target region is fluorescence.

52. The method according to claim 50, wherein the 16/18 probe emits a fluorescent signal at a wavelength different from that of the pan-HPV probe.

53. The method according to claim 47, wherein the primers comprise one or both of the following primer pairs:
a) for HPV16 SEQ ID NO: 19 and SEQ ID NO: 20
b) for HPVI8 SEQ ID NO: 21 and SEQ ID NO: 22.

54. The method according to claim 39, further comprising
conducting a positive control for the real time PCR, comprising
combining the sample with primers for real time PCR amplification of a control DNA sequence preferably human DNA, present in the sample, and a labelled control probe capable of emitting a signal upon amplification of the control DNA sequence preferably human DNA,
providing conditions for real time PCR, and
measuring for a signal from the control probe,
wherein detection of the signal indicates that the real time PCR is functional.

55. The method according to claim 39, wherein the sample is a liquid based cytology sample from a human cervical smear.

56. The composition comprising a labelled nucleic acid probe comprising nucleotide sequence ACTTTCGTTT (SEQ ID NO: 25) or the reverse complement thereof and a detectable label.

57. The composition according to claim 56, wherein the probe comprises LNA nucleotides.

58. The composition according to claim 56, wherein the probe is a 10-nucleotide molecule of sequence consisting of ACTTTCGTTT (SEQ ID NO: 25) or the reverse complement thereof labelled with a fluorophore and a quencher.

59. The composition according to claim 56, wherein the probe is labelled at its 5′ end with a fluorophore and at its 3′ end with a quencher.

60. Use of a composition according to claim 56 in performing real time PCR for detection of HPV.
